(19) United States
(12) Patent Application Publication
(10) Pub. No.: US 2017/0368192 A1
(43) Pub. Date: Dec. 28, 2017

(54) LUMINAL VESSEL COATING FOR ARTERIOVENOUS FISTULA

(71) Applicant: Symic IP, LLC, Emeryville, CA (US)

(72) Inventors: John Eric Paderi, San Francisco, CA (US); Katherine Allison Stuart, San Francisco, CA (US); Alyssa Panitch, Davis, CA (US); Nathan Bachtell, San Francisco, CA (US); Kenneth Horne, Emeryville, CA (US)

(21) Appl. No.: 15/518,720

(22) PCT Filed: Oct. 13, 2015
(86) PCT No.: PCT/US2015/055399

§ 371 Date: Apr. 12, 2017

Related U.S. Application Data
(60) Provisional application No. 62/063,332, filed on Oct. 13, 2014.

(51) Int. Cl. A61K 47/64; A61K 9/08; A61K 31/726; A61L 27/50; A61L 27/34; A61L 33/12; A61F 2/06; A61L 27/54; A61L 33/00; A61K 31/727

(52) U.S. Cl. CPC A61K 47/64; A61L 33/0017; A61K 9/08; A61K 31/726; A61K 31/727; A61L 27/34; A61L 33/128; A61L 33/0094; A61F 2/064; A61L 27/54; A61L 27/507; A61L 2300/236; A61L 2300/42; A61L 2300/606; A61L 2420/02; A61L 2430/22

(57) ABSTRACT

This disclosure provides a method for improving maturation of an arteriovenous fistula (AVF) in a patient in need of hemodialysis, which method entails applying a solution to the internal wall of a lumen of an AVF; and restoring or initiating blood flow in the AVF, wherein the solution comprises an effective amount of a synthetic proteoglycan comprises a glycan having from about 1 to about 80 collagen-binding peptide(s) bonded to the glycan. Also provided are methods for preparing a vascular graft for a bypass surgery, comprising contacting the internal wall of a section of a blood vessel with a solution comprising an effective amount of the synthetic proteoglycan.

### CROSS REFERENCE TO RELATED APPLICATION

This application claims the benefit under 35 U.S.C. 119(e) to U.S. Provisional Application Ser. No. 62/063,332, filed on Oct. 13, 2014, the entirety of which is incorporated herein by reference.

### STATEMENT AS TO FEDERALLY SPONSORED RESEARCH OR DEVELOPMENT

This invention was made with government support under 1R41DK100156-01 awarded by the National Institutes of Health. The government has certain rights in the invention.

### FIELD

This disclosure provides compositions and methods for improving maturation, improving patency, enlarging inner diameter of the blood vessels, in particular, the veins, and reducing stenosis and neointimal hyperplasia of a vascular access, such as a native arteriovenous fistulas (AVF) in patients in need of hemodialysis.

### BACKGROUND

More than 20 million American adults (1 in 10) have some level of chronic kidney disease, with a growing incidence in the aging population. At the end of 2009, nearly 900,000 patients were being treated for end-stage renal disease (ESRD). Nearly 400,000 ESRD patients receive some form of dialysis, with the vast majority of hemodialysis being performed in a clinic. One of the major causes of morbidity for the ESRD population is hemodialysis vascular access dysfunction, which is responsible for 20% of all hospitalizations for this population. Vascular access accounts for 7.5% of Medicare's spending on the ESRD programs, a total of over $1 billion per year. In the past 3 decades, there have been no major advances in the field of hemodialysis vascular access, resulting in a huge unmet clinical need.

There are three main forms of hemodialysis vascular access: native arteriovenous fistula (AVF), polytetrafluoroethylene (PTFE) graft, and the cuffed double-lumen silicone catheter. The native AVF has the lowest rates of infection and thrombosis once it has fully matured, and is thus the preferred method of access. Additionally, native AVF costs $10,000 less annually per patient than a PTFE graft. The “Fistula First” initiative by the Centers of Medicare and Medicaid Services has set a goal to increase the use of AV fistulas for hemodialysis access to greater than 50% for new patients, and to greater than 40% for those already on hemodialysis.

Although native AVF is the preferred method of access, hemodialysis patients receiving a fistula have two primary clinical needs: improvement in maturation rates, and improvement in overall patency rates. The failure of fistulas to mature without additional intervention is approximately 37-50%. For those fistulas that do mature, the failure rate at 12 months is approximately 50%, and additional interventions are often necessary to restore patency. Both early and late failures of AVF are characterized by vascular stenosis, and the classical histological lesion associated with all AVF failure is neointimal hyperplasia. Presence of neointimal hyperplasia can be seen as early as 3.5 months after AVF placement. The cause of failure is not clear, but multiple factors increase risk of failure, including size of artery and/or vein, surgical manipulation, hemodynamic stressors, genetic predispositions to vasoconstriction, and neointimal hyperplasia due to endothelial and smooth muscle cell injury.

Neointimal hyperplasia is central to hemodialysis vascular access dysfunction, and has been described as the Achilles' heel of hemodialysis. Access thrombosis causes 80% of all vascular access dysfunction, and in more than 90% of thrombosed grafts and fistulae the underlying pathology is a stenosis caused by venous neointimal hyperplasia at either the venous anastomotic site or in the proximal vein. While many factors, as noted above, play a role in the development of neointimal hyperplasia, the extent of damage to the endothelial layer of a vessel has been directly related to the degree of neointimal hyperplasia that occurs. It is now appreciated that all vascular manipulation results in damage to the vessel, particularly to the fragile endothelial cells lining the lumen. Damage to the endothelium exposes underlying collagen in the vessel. Platelets are well adapted to bind to collagen, where they become activated and release or upregulate numerous vasoactive agents and factors that induce coagulation and inflammation. The body has evolved these responses as an effective means for controlling blood loss and fighting injection following injury; however this same collagen initiated coagulation and inflammatory response occurring inside a vessel is the initial step in the pathways that result in thrombosis and neointimal hyperplasia.

### SUMMARY

The present disclosure provides compositions and methods for addressing the injury that occurs during the creation of arteriovenous fistulas (AVF) or other vein grafts. A localized treatment is disclosed using a synthetic polymeric luminal coating, which binds specifically to exposed collagen, where it blocks platelet adhesion to the vessel wall and thus inhibits the initiating events in thrombosis and intimal hyperplasia. Additionally, the coating promotes rapid re-endothelialization of the vessel wall, resulting in faster healing. Experimental data showed that application of the coating to native AV fistulas during the creation resulted in fistulas with significantly less stenosis and larger diameters.

More generally, the disclosed compositions and methods are useful for establishing a vascular access in a patient which method can entail applying a solution of the disclosure to a wall of a blood vessel in a vascular access; and restoring or initiating blood flow in the vascular access. In some aspects, the wall is an internal wall of the blood vessel, but it can also be the external wall of any blood vessel.

In some aspects, the vascular access is an arteriovenous fistula (AVF), an arteriovenous graft (AVG), or a durable vascular access used for parenteral nutrition, chemotherapy, or plasmapheresis. It is contemplated that the solution reduces exposure of the wall to platelets. In some aspects, the wall comprises a cell or tissue exposed to blood flow due to injury or a surgical procedure. It is shown that application of the solution improves patency, improves survival, improves blood flow, enlarge vascular inner diameter, or reduce stenosis in the vascular access, such as AVF and AVG.

In one embodiment, the present disclosure provides a method for improving maturation of an arteriovenous fistula (AVF) in a patient in need of hemodialysis, or alternatively for improving patency, enlarging inner diameter of the veins, reducing stenosis, reducing neointimal hyperplasia, reducing hemodynamic stress, reducing endothelial or smooth muscle cell injury, reducing vascular access dysfunction, reducing coagulation or inflammation at the AVF. In some aspects, the method entails applying a solution to the internal wall of a lumen of an AVF; and restoring or initiating blood flow in the AVF, wherein the solution comprises an effective amount of a synthetic peptidoglycan (or proteoglycan) of the present disclosure.

It is contemplated that the synthetic proteoglycans may be administered to the interior of the patient's vessel percutaneously or intravenously. The percutaneous or intravenous delivery allows for treatment of a patient post-surgical fistula creation. The synthetic proteoglycans may be delivered for treatment of the vessel, maintenance of the vessel, or for prevention of the failure of the fistula.

In some embodiments, the methods further include carrying out one or more maintenance applications, such as balloon-assisted maturation, balloon angioplasty, or declotting procedures. Still, in some embodiments, prophylactic delivery at the time of hemodialysis, especially following the procedure when especially high flow rates damage the endothelium and an injection in the graft or fistula is contemplated to be beneficial for maintenance and prevention of stenosis.

In one embodiment, the present disclosure provides a method for preparing a vascular graft for a bypass surgery, comprising contacting the internal wall of a section of a blood vessel with a solution of the present disclosure.

In one embodiment, the present disclosure provides a vascular graft comprising a section of a blood vessel comprising an internal wall bound to an effective amount of a synthetic proteoglycan of the present disclosure. Methods of using vascular grafts are also provided.

In one embodiment, a method is provided for preventing or reducing graft failure in a patient undergoing a bypass grafting procedure, comprising implanting the graft into the circulation system of the patient.

In another embodiment, the present disclosure provides a method for preventing or reducing graft failure in a patient undergoing a bypass grafting procedure, comprising implanting a graft into the circulation system of the patient, and injecting into the inside of the graft, before, during or following the implantation, a solution of the present disclosure.

### BRIEF DESCRIPTION OF THE DRAWINGS

FIG. 1A-B show the delivery of DS-SILY, a proteoglycan described herein, to the fistula, and the fistula after closure and restoration of blood flow. FIG. 1A, A single suture was left open when the AVF was created, and the DS-SILY solution was flushed through the clamped fistula. FIG. 1B, The fistula was closed and blood flow was restored.

FIG. 2A-C show that, in all three delivery methods (A: method 1; B: method 2; C: method 3), labeled DS-SILY (shown as white) was evident on the luminal surface of the vein. Green staining (phalloidin; shown as gray) shows a lack of cells at the surface, indicating endothelial cell damage during the procedure.

FIG. 3 is CT image revealing smaller diameter or occluded veins of control treated vessels, while DS-SILY treated vessels remained open.

FIG. 4A-B summarize the data from FIG. 3. In FIG. 4A, the smallest measured diameter in the fistulas was plotted, with a control vessels having an average of 2.1 mm diameters vs 6.3 mm diameters for DS-SILY treated vessels (n=4, p=0.019). In FIG. 4B, paired data show the smallest diameters of the fistula in each animal to account for inter-animal variability. In all cases, the fistula treated with DS-SILY has a larger diameter than the control fistula.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a peptide” includes a plurality of peptides.

### 1. Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The following abbreviations used herein have the following meanings.

| | |
|---|---|
| ° C. | Degrees Celsius |
| μg | Microgram |
| BID | Administered Twice Daily |
| BMPH | N-[β-maleimidopropionic acid]hydrazide |
| BMPH-CS | BMPH Linker-Chondroitin Sulfate Conjugate |
| CD44 | Cell-Surface Glycoprotein CD44 Antigen |
| cps | Centipoise |
| CS | Chondroitin sulfate |
| Dex | Dextran |
| DNA | Deoxyribonucleic acid |
| DS | Dermatan Sulfate |
| ECM | Extracellular Matrix |
| EDTA | Ethylenediaminetetraacetic Acid |
| ELISA | Enzyme-Linked Immunosorbent Assay |
| FGF | Fibroblast Growth Factor |
| Fmoc | 9-Fluorenylmethoxycarbonyl |
| GAG | Glycosaminoglycan |
| Hep | Heparin |
| HEPES | 2-[4-(2-hydroxyethyl)piperazin-1-yl]ethanesulfonic acid |
| HLB | Hydrophile/Lipophile/Balance |
| HPC | Hydroxyl Propylcellulose |
| HPMC | Hydroxypropylmethyl Cellulose |
| ITC | Isothermal Titration Calorimeters |
| kDa | KiloDalton |
| kg | Kilogram |
| MES | 2-ethanesulfonic acid |
| mg | Milligram |
| mL | Milliliter |
| MOPS | 3-(N-morpholino)propanesulfonic acid |
| mOsm | Milliosmole |
| mV | Millivolt |
| ng | Nanogram |
| PBS | Phosphate buffered saline |
| PDPH | 3-(2-pyridyldithio)propionyl hydrazide |
| PIPES | piperazine-N,N′-bis(2-ethanesulfonic acid) |
| QD | Administered Once Daily |
| SILY | RRANAALKAGELYKSILY (SEQ ID NO: 1) |
| SPR | Surface Plasmon Resonance |
| TAPS | 3-[[1,3-dihydroxy-2-(hydroxymethyl)propan-2- |
| | yl]amino]propane-1-sulfonic acid |
| TES | 2-[[1,3-dihydroxy-2-(hydroxymethyl)propan-2- |
| | yl]amino]ethanesulfonic acid |
| Tris | 2-Amino-2-hydroxymethyl-propane-1,3-diol |
| w/w | Weight/Weight |
| w/v | Weight/Volume |

As used herein, the term “treating and/or preventing” refers to preventing, curing, reversing, attenuating, alleviating, minimizing, suppressing or halting vascular access dysfunction during or after AVF.

As used herein, the term “patient” refers to a subject having renal failure, in need of hemodialysis or otherwise in need of high venous blood flow or durable vascular access, such as those undergoing total parenteral nutrition, chemotherapy, or plasmapheresis.

As used herein, the term “administering” or “administration” refers to the delivery of one or more therapeutic agents to a patient.

As used herein, the term “amino acid” refers to either a natural and/or unnatural or synthetic amino acid, including glycine and both the D and L optical isomers, amino acid analogs and peptidomimetics.

A “peptide” is a chain of two or more amino acid monomers linked by peptide (amide) bonds. A peptide of three or more amino acids is commonly called an oligopeptide if the peptide chain is short. If the peptide chain is long, the peptide is commonly called a polypeptide or a protein. As used herein, the term “peptide” is intended to refer a linear or branched chain of amino acids linked by peptide (or amide) bonds. In one embodiment, the peptide comprises from about 3 to about 120 amino acids, or from about 3 to about 110 amino acids, or from about 3 to about 100 amino acids, or from about 3 to about 90 amino acids, or from about 3 to about 80 amino acids, or from about 3 to about 70 amino acids, or from about 3 to about 60 amino acids, or from about 3 to about 50 amino acids, or from about 3 to about 40 amino acids, or from about 5 to about 120 amino acids, or from about 5 to about 100 amino acids, or from about 5 to about 90 amino acids, or from about 5 to about 80 amino acids, or from about 5 to about 70 amino acids, or from about 5 to about 60 amino acids, or from about 5 to about 50 amino acids, or from about 5 to about 40 amino acids, or from about 5 to about 30 amino acids, or from about 5 to about 20 amino acids, or from about 5 to about 10 amino acids.

As used herein, the terms “peptidoglycan,” “proteoglycan,” “proteoglycan mimetic,” and “synthetic proteoglycan” are used interchangeably and refer to a synthetic conjugate that comprises a glycan and one or more synthetic peptides covalently bonded thereto. The glycan portion can be made synthetically or derived from animal sources. The synthetic peptides can be covalently bonded directly to the glycan or via a linker. For methods of conjugating collagen-binding peptides to glycans, see, e.g., US 2013/0190246, US 2012/0100106, and US 2011/0020298, the disclosures of which are incorporated herein by reference in their entirety. In some embodiments, the term synthetic proteoglycan includes proteoglycans. In one embodiment, the molecular weight range for the synthetic proteoglycan is from about 13 kDA to about 1.2 MDa, or from about 500 kDa to about 1 MDa, or from about 20 kDa to about 90 kDa, or from about 10 kDa to about 70 kDa.

As used herein, the term “glycan” refers to a compound having a large number of monosaccharides linked glycosidically. In certain embodiments, the glycan is a glycosaminoglycan (GAG), which comprise 2-aminosugars linked in an alternating fashion with uronic acids, and include polymers such as heparin, heparan sulfate, chondroitin, keratin, and dermatan. Accordingly, non-limiting examples of glycans which can be used in the embodiments described herein include alginate, agarose, dextran (Dex), chondroitin, chondroitin sulfate (CS), dermatan, dermatan sulfate (DS), heparan sulfate, heparin (Hep), keratin, keratan sulfate, and hyaluronic acid (HA). In one embodiment the molecular weight of the glycan is a key parameter in its biological function. In another embodiment the molecular weight of the glycan is varied to tailor the effects of the synthetic proteoglycan mimic (see e.g., Radek, K. A., et al., Wound Repair Regen., 2009, 17: 118-126; and Taylor, K. R., et al., J. Biol. Chem., 2005, 280:5300-5306). In another embodiment, the glycan molecular weight is about 46 kDa. In another embodiment, the glycan is degraded by oxidation and alkaline elimination (see e.g., Fransson, L. A., et al., Eur. J. Biochem., 1980, 106:59-69) to afford degraded glycan having a lower molecular weight (e.g., from about 10 kDa to about 50 kDa). In some embodiments, the glycan is unmodified.

In one embodiment, the GAG is heparin. In one embodiment, the GAG is dermatan sulfate (DS). The biological functions of DS are extensive, and include the binding and activation of growth factors FGF-2, FGF-7, and FGF-10, which promote endothelial cell and keratinocyte proliferation and migration. In one embodiment, the DS molecular weight is about 46 kDa. In another embodiment, the DS is degraded by oxidation and alkaline elimination (see e.g., Fransson, L. A., et al., Eur. J. Biochem., 1980, 106:59-69) to afford degraded DS having a low molecular weight (e.g., 10 kDa). In some embodiments, the weight range of the DS is from about 10 kDa to about 70 kDa.

As used herein, the terms “bonded” and “covalently bonded” can be used interchangeably, and refer to the sharing of one or more pairs of electrons by two atoms. In one embodiment, the peptide is bonded to the glycan. In one embodiment the peptide is covalently bonded to the glycan, with or without a linker. In one embodiment the peptide is covalently bonded to the glycan via a linker. In one embodiment the peptide is directly bonded to the glycan.

In one embodiment, the synthetic proteoglycans of the disclosure bind, either directly or indirectly to collagen. The terms “binding” or “bind” as used herein are meant to include interactions between molecules that may be detected using, for example, a hybridization assay, surface plasmon resonance, ELISA, competitive binding assays, isothermal titration calorimetry, phage display, affinity chromatography, rheology or immunohistochemistry. The terms are also meant to include “binding” interactions between molecules. Binding may be “direct” or “indirect”. “Direct” binding comprises direct physical contact between molecules. “Indirect” binding between molecules comprises the molecules having direct physical contact with one or more molecules simultaneously. This binding can result in the formation of a “complex” comprising the interacting molecules. A “complex” refers to the binding of two or more molecules held together by covalent or non-covalent bonds, interactions or forces.

As used herein, the term “collagen-binding peptide” refers to an optionally synthetic peptide comprising a collagen-binding sequence (or domain or unit). “Collagen binding” indicates an interaction with collagen that could include hydrophobic, ionic (charge), and/or Van der Waals interactions, such that the compound binds or interacts favorably with collagen. This binding (or interaction) is intended to be differentiated from covalent bonds and nonspecific interactions with common functional groups, such that the collagen-binding peptide would interact with any species containing that functional group to which the peptide binds on the collagen. See, e.g., Li, Y., et al., Current Opinion in Chemical Biology, 2013, 17: 968-975, Helmes, B. A., et al., J. Am. Chem. Soc. 2009, 131, 11683-11685, and Petsalaki, E., et al., PLoS Comput Biol, 2009, 5(3): e1000335.

In one aspect, the collagen-binding peptide binds to one or more of collagen type I, II, III, IV, V, VI, VII, VIII, IX, X, XI, XII, XIIII, or XIV. In one aspect, the collagen biding peptide promotes or inhibits fibrillogenesis upon binding to collagen. In one aspect, the collagen biding peptide does not promote or inhibit fibrillogenesis upon binding to collagen. In some embodiments, the peptide binds to type I collagen. In other embodiments, the peptide binds to type IV collagen. In certain embodiments, one or more peptide(s) having a specified binding affinity for collagen can be used in the synthetic proteoglycans described herein. For example, the synthetic proteoglycans can comprise at least one peptide which has binding affinity to type I collagen and at least one peptide which has binding affinity to type IV collagen. In another embodiment, the peptides have binding affinity to type I collagen. In another embodiment, the peptides have binding affinity to type IV collagen. In certain embodiments, the peptides have binding affinity to type II collagen. In certain embodiments, the peptides have binding affinity to type III collagen. In certain embodiments, the peptide binds to more than one type of collagen, where the relative affinity to each collagen type may vary.

Further, the peptides as used herein may comprise more than one binding unit, where the binding unit can be the same or different. For example, in certain embodiments, the peptide comprises two or more collagen-binding units, where the collagen-binding units are the same. In another embodiment, the peptide comprises two or more collagen-binding units, where the collagen-binding units are different.

The collagen-binding peptide can have amino acid homology with a portion of a protein normally or not normally involved in collagen fibrillogenesis. In one embodiment, the collagen-binding peptide comprises from about 5 to about 40 amino acids, or from about 5 to about 20 amino acids, or from about 5 to about 10 amino acids. In some embodiments, these peptides have homology or sequence identity to the amino acid sequence of a small leucine-rich proteoglycan, a platelet receptor sequence, or a protein that regulates collagen fibrillogenesis. In various embodiments, the collagen-binding peptide comprises an amino acid sequence selected from:

i)
(SEQ ID NO: 1)
RRANAALKAGELYKSILY,
(SEQ ID NO: 2)
RLDGNEIKR,
(SEQ ID NO: 3)
AHEEISTTNEGVM,
(SEQ ID NO: 4)
GELYKSILY,
(SEQ ID NO: 5)
NGVFKYRPRYFLYKHAYFYPPLKRFPVQ,
(SEQ ID NO: 6)
CQDSETRTFY,
(SEQ ID NO: 7)
TKKTLRT,
(SEQ ID NO: 8)
GLRSKSKKFRRPDIQYPDATDEDITSHM,
(SEQ ID NO: 9)
SQNPVQP,
(SEQ ID NO: 10)
SYIRIADTNIT,
(SEQ ID NO: 11)
KELNLVYT,
(SEQ ID NO: 12)
GSITTIDVPWNVGC,
(SEQ ID NO: 13)
GSITTIDVPWNV;
(SEQ ID NO: 14)
RRANAALKAGELYKCILY,
(SEQ ID NO: 15)
GELYKCILY,
(SEQ ID NO: 16)
GQLYKSILY,
or
(SEQ ID NO: 17)
RRANAALKAGQLYKSILY

or ii) a peptide comprising a sequence with at least about 80% sequence identity to the amino acid sequence of i) and capable of binding to collagen.

In certain embodiments, the collagen-binding peptide comprises an amino acid sequence that has at least about 80%, or at least about 83%, or at least about 85%, or at least about 90%, or at least about 95%, or at least about 98%, or at least about 100% sequence identity with the collagen-binding unit(s) of the von Willebrand factor (vWF) or a platelet collagen receptor as described in Chiang, T. M., et al. J. Biol. Chem., 2002, 277: 34896-34901, Huizinga, E. G. et al., Structure, 1997, 5: 1147-1156, Romijn, R. A., et al., J. Biol. Chem., 2003, 278: 15035-15039, and Chiang, et al., Cardio. & Haemato. Disorders-Drug Targets, 2007, 7: 71-75, each incorporated herein by reference. A non-limiting example is WREPSFCALS (SEQ ID NO: 18), derived from vWF.

Various methods for screening peptide sequences for collagen-binding affinity (or a collagen-binding unit) are routine in the art. Other peptide sequences shown to have collagen-binding affinity (or a collagen-binding unit) which can be used in the synthetic proteoglycans and methods disclosed herein include but are not limited to, βAWHCTTKFPHHYCLYBip (SEQ ID NO: 19), βAHKCPWHLYTTHYCFTBip (SEQ ID NO: 20), βAHKCPWHLYTHYCFT (SEQ ID NO: 21), etc., where Bip is biphenylalanine and βA is beta-alanine (see, Abd-Elgaliel, W. R., et al., Biopolymers, 2013, 100(2), 167-173), GROGER (SEQ ID NO: 22), GMOGER (SEQ ID NO: 23), GLOGEN (SEQ ID NO: 24), GLOGER (SEQ ID NO: 25), GLKGEN (SEQ ID NO: 26), GFOGERGVEGPOGPA (SEQ ID NO: 27), etc., where O is 4-hydroxyproline (see, Raynal, N., et al., J. Biol. Chem., 2006, 281(7), 3821-3831), HVWMQAPGGGK (SEQ ID NO: 28) (see, Helms, B. A., et al., J. Am. Chem. Soc. 2009, 131, 11683-11685), WREPSFCALS (SEQ ID NO: 18) (see, Takagi, J., et al., Biochemistry, 1992, 31, 8530-8534), WYRGRL (SEQ ID NO: 29), etc. (see, Rothenfluh D. A., et al., Nat Mater. 2008, 7(3), 248-54), WTCSGDEYTWHC (SEQ ID NO: 30), WTCVGDHKTWKC (SEQ ID NO: 31), QWHCTTRFPHHYCLYG (SEQ ID NO: 32), etc. (see, U.S. 2007/0293656), STWTWNGSAWTWNEGGK (SEQ ID NO: 33), STWTWNGTNWTRNDGGK (SEQ ID NO: 34), etc. (see, WO/2014/059530), CVWLWEQC (SEQ ID NO: 35) (see, Depraetere H., et al., Blood. 1998, 92, 4207-4211; and Duncan R., Nat Rev Drug Discov, 2003, 2(5), 347-360), CMTSPWRC (SEQ ID NO: 36), etc. (see, Vanhoorelbeke, K., et al., J. Biol. Chem., 2003, 278, 37815-37821), CPGRVMHGLHLGDDEGPC (SEQ ID NO: 37) (see, Muzzard, J., et al., PLoS one. 4 (e 5585) I-10), KLWLLPK (SEQ ID NO: 38) (see, Chan, J. M., et al., Proc Natl Acad Sci U.S.A., 2010, 107, 2213-2218), and CQDSETRTFY (SEQ ID NO: 6), etc. (see, U.S. 2013/0243700), wherein each is hereby incorporated by reference in its entirety. Additional peptide sequences shown to have collagen-binding affinity (or a collagen-binding unit) which can be used in the synthetic proteoglycans and methods disclosed herein include but are not limited to, LSELRLHEN (SEQ ID NO: 39), LTELHLDNN (SEQ ID NO: 40), LSELRLHNN (SEQ ID NO: 41), LSELRLHAN (SEQ ID NO: 42), LRELHLNNN (SEQ ID NO: 43) (see, Fredrico, S., Angew. Chem. Int. Ed. 2015, 37, 10980-10984).

In certain embodiments, the peptides include one or more sequences selected from the group consisting of RVMHGLHLGDDE (SEQ ID NO: 44), D-amino acid EDDGLHLGHMVR (SEQ ID NO: 45), RVMHGLHLGNNQ (SEQ ID NO: 46), D-amino acid QNNGLHLGHMVR (SEQ ID NO: 47), RVMHGLHLGNNQ (SEQ ID NO: 48), GQLYKSILYGSG-4K2K (SEQ ID NO: 49) (a 4-branch peptide), GSGQLYKSILY (SEQ ID NO: 50), GSGGQLYKSILY (SEQ ID NO: 51), KQLNLVYT (SEQ ID NO: 52), CVWLWQQC (SEQ ID NO: 53), WREPSFSALS (SEQ ID NO: 54), GHRPLDKKREEAPSLRPAPPPISGGGYR (SEQ ID NO: 55), and GHRPLNKKRQQAPSLRPAPPPISGGGYR (SEQ ID NO: 56).

As used herein, the term “sequence identity” refers to a level of amino acid residue or nucleotide identity between two peptides or between two nucleic acid molecules. When a position in the compared sequence is occupied by the same base or amino acid, then the molecules are identical at that position. A peptide (or a polypeptide or peptide region) has a certain percentage (for example, at least about 60%, or at least about 65%, or at least about 70%, or at least about 75%, or at least about 80%, or at least about 83%, or at least about 85%, or at least about 90%, or at least about 95%, or at least about 98% or at least about 99%) of “sequence identity” to another sequence means that, when aligned, that percentage of bases (or amino acids) are the same in comparing the two sequences. It is noted that, for any sequence (“reference sequence”) disclosed in this application, sequences having at least about 60%, or at least about 65%, or at least about 70%, or at least about 75%, or at least about 80%, or at least about 83%, or at least about 85%, or at least about 90%, or at least about 95%, or at least about 98% or at least about 99% sequence identity to the reference sequence are also within the disclosure. Likewise, the present disclosure also includes sequences that have one, two, three, four, or five substitution, deletion or addition of amino acid residues or nucleotides as compared to the reference sequences.

In any of the embodiments described herein, any one or more of the synthetic peptides (e.g., the collagen-binding peptide) may have a spacer sequence comprising from one to about five amino acids. It is contemplated that any amino acid, natural or unnatural, can be used in the spacer sequence, provided that the spacer sequence does not significantly interfere with the intended binding of the peptide. Exemplary spacers include, but are not limited to, short sequences comprising from one to five glycine units (e.g., G, GG, GGG, GGGG, or GGGGG), optionally comprising cysteine (e.g., GC, GCG, GSGC, or GGC) and/or serine (e.g., GSG, or GSGSG), or from one to five arginine units (e.g., R, RR, RRR, etc.). The spacer can also comprise non-amino acid moieties, such as polyethylene glycol (PEG), 6-aminohexanoic acid, or combinations thereof, with or without an amino acid spacer. The spacer can be attached to either the C-terminus or the N-terminus of the peptide to provide a point of attachment for a glycan or a glycan-linker conjugate. In certain embodiments, the spacer comprises more than one binding site (may be linear or branched) such that more than one peptide sequence can be bound thereto, thus creating a branched construct. The binding sites on the spacer can be the same or different, and can be any suitable binding site, such as an amine or carboxylic acid moiety, such that a desired peptide sequence can be bound thereto (e.g. via an amide bond). Thus in certain embodiments, the spacer contains one or more lysine, glutamic acid or aspartic acid residues. Such constructs can provide peptides having more than one collagen-binding unit of the formula PₙL, where P is a collagen-binding unit, L is a spacer and n is an integer from 2 to about 10, or from 2 to 8, or from 2 to 6, or from 2 to 5, or from 2 to 4, or 2, or 3, or 4, or 5, or 6, or 7, or 8, or 9, or 10. For example, the spacer L can be an amino acid sequence such as KGSG, KKGSG, or KKKGSG, etc., providing 2, 3, or 4 binding sites, respectively. Exemplary collagen-binding constructs include, but are not limited to, (GELYKSILYGSG)₂KGSG (SEQ ID NO: 57), (GELYKSILYGSG)₃KKGSG (SEQ ID NO: 58), (GELYKSILYGSG)₄KKKGSG (SEQ ID NO: 59), (GQLYKSILYGSG)₂KGSG (SEQ ID NO: 60), (GQLYKSILYGSG)₃KKGSG (SEQ ID NO: 61), and (GQLYKSILYGSG)₄KKKGSG (SEQ ID NO: 62).

Accordingly, in certain embodiments, the synthetic peptide is RYPISRPRKRGSG (SEQ ID NO: 63), RRANAALKAGELYKSILYGC (SEQ ID NO: 64), or GELYKSILYGC (SEQ ID NO: 65).

In any of the embodiments described herein, a synthetic peptide (e.g., a collagen-binding peptide) comprises any amino acid sequence described in the preceding paragraph or an amino acid sequence having at least about 80%, or at least about 83%, or at least about 85%, or at least about 90%, or at least about 95%, or at least about 98%, or at least about 100% homology to any of these amino acid sequences. In various embodiments, the peptide components of the synthetic proteoglycan described herein can be modified by the inclusion of one or more conservative amino acid substitutions. As is well-known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in Table 1.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu, Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, |
| | L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

As used herein, the term “extracellular matrix” refers to the extracellular part of tissue that provides structural and biochemical support to the surrounding cells.

As used herein, the term “linker” refers to chemical bond, atom, or group of atoms that connects two adjacent chains of atoms in a large molecule such as a peptide, synthetic proteoglycan, protein or polymer. In various embodiments, the linker comprises two or more chemically orthogonal functionalities on a rigid scaffold (e.g., any suitable bifunctional linker, such as N-[β-maleimidopropionic acid]hydrazide (BMPH), 3-(2-pyridyldithio)propionyl hydrazide (PDPH)), or the peptide GSG.

As used herein, the term “composition” refers to a preparation suitable for administration to an intended patient for therapeutic purposes that contains at least one pharmaceutically active ingredient, including any solid form thereof. The composition may include at least one pharmaceutically acceptable component to provide an improved formulation of the compound, such as a suitable carrier. In certain embodiments, the composition is formulated as a film, gel, patch, or liquid solution. As used herein, the term topically refers to administering a composition non-systemically to the surface of a tissue and/or organ (internal or, in some cases, external) to be treated, for local effect.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the internal surface of a vein.

As used herein, the term “formulated” or “formulation” refers to the process in which different chemical substances, including one or more pharmaceutically active ingredients, are combined to produce a dosage form. In certain embodiments, two or more pharmaceutically active ingredients can be coformulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “delivery” refers to approaches, formulations, technologies, and systems for transporting a pharmaceutical composition in the body as needed to safely achieve its desired therapeutic effect. In some embodiments, an effective amount of the composition is formulated for delivery into the AVF or a vein graft of a patient.

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

As used herein, the term “polymer matrix” or “polymeric agent” refers to a biocompatible polymeric materials. The polymeric material described herein may comprise, for example, sugars (such as mannitol), peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

As used herein, the term “absorbable” refers to the ability of a material to be absorbed into the body. In certain embodiments, the polymeric matrix is absorbable, such as, for example collagen, polyglycolic acid, polylactic acid, polydioxanone, and caprolactone. In other embodiments, the polymer is non-absorbable, such as, for example polypropylene, polyester or nylon.

As used herein, the term “pH buffering agent” refers to an aqueous buffer solution which resists changes in pH when small quantities of acid or base are added to it. pH Buffering solutions typically comprise of a mixture of weak acid and its conjugate base, or vice versa. For example, pH buffering solutions may comprise phosphates such as sodium phosphate, sodium dihydrogen phosphate, sodium dihydrogen phosphate dihydrate, disodium hydrogen phosphate, disodium hydrogen phosphate dodecahydrate, potassium phosphate, potassium dihydrogen phosphate and dipotassium hydrogen phosphate; boric acid and borates such as, sodium borate and potassium borate; citric acid and citrates such as sodium citrate and disodium citrate; acetates such as sodium acetate and potassium acetate; carbonates such as sodium carbonate and sodium hydrogen carbonate, etc. pH Adjusting agents can include, for example, acids such as hydrochloric acid, lactic acid, citric acid, phosphoric acid and acetic acid, and alkaline bases such as sodium hydroxide, potassium hydroxide, sodium carbonate and sodium hydrogen carbonate, etc. In some embodiments, the pH buffering agent is a phosphate buffered saline (PBS) solution (i.e., containing sodium phosphate, sodium chloride and in some formulations, potassium chloride and potassium phosphate).

As used herein, the term “concurrently” refers to simultaneous (i.e., in conjunction) administration. In one embodiment, the administration is coadministration such that two or more pharmaceutically active ingredients, including any solid form thereof, are delivered together at one time.

As used herein, the term “sequentially” refers to separate (i.e., at different times) administration. In one embodiment, the administration is staggered such that two or more pharmaceutically active ingredients, including any solid form thereof, are delivered separately at different times.

The present disclosure, in one embodiment, provides a new approach to address the unmet need of vascular access dysfunction in patients receiving hemodialysis. In one aspect, the approach entails generation of a luminal vessel coating designed from a synthetic collagen-binding proteoglycan. These engineered molecules locally bind to exposed collagen through physical peptide-collagen interactions. When bound to collagen, the synthetic proteoglycan has a number of functions including 1) acting as a barrier to platelet attachment/activation, 2) protecting collagen from degradation by inhibiting MMP access, and 3) sequestering growth factors FGF-2, FGF-7, and FGF-10, thus promoting endothelial and epithelial cell proliferation and migration.

The collagen-binding synthetic proteoglycan, in one embodiment, includes a polysaccharide backbone with covalently attached collagen-binding peptides. The synthetic proteoglycans can compete for platelet binding sites on collagen and prevent platelet binding and activation. The glycan backbone can be negatively charged and bind water molecules, creating a hydrophilic barrier over the collagen surface that prevents platelet and protein adhesion. By masking the exposed collagen, rather than inhibiting normal platelet function, the synthetic proteoglycan can provide a local treatment that addresses the initial steps in the cascade to inflammation and intimal hyperplasia.

In AVF, the neointimal hyperplasia mostly occurs in the venous portion of the AVF. While the initial mechanisms of intimal hyperplasia are similar in arteries and veins, there are differences in the resulting lesions. Venous neointimal hyperplasia tends to be a more aggressive lesion than arterial intimal hyperplasia in the setting of peripheral vascular disease, and have poorer response to angioplasty. The ability of the disclosed synthetic proteoglycan to prevent platelet binding and intimal hyperplasia in an arterial injury, is contemplated to contribute to its ability to reduce or prevent neointimal hyperplasia.

Thus, in some embodiments, the present disclosure provides a method for improving maturation of an arteriovenous fistula (AVF) in a patient in need of hemodialysis, or alternatively for improving patency, enlarging inner diameter of the veins, reducing stenosis, reducing neointimal hyperplasia, reducing hemodynamic stress, reducing endothelial or smooth muscle cell injury, reducing vascular access dysfunction, reducing coagulation or inflammation at the AVF. In some aspects, the method entails applying a solution to the internal wall of a lumen of an AVF; and restoring or initiating blood flow in the AVF, wherein the solution comprises an effective amount of a synthetic proteoglycan of the present disclosure.

In some aspects, for a newly created AVF before blood flow is initiated, the solution is applied less than about 10 minutes before the blood flow is initiated. In some aspects, the solution is applied less than about 20, 15, 10, 9, 8, 7, 6, 5, 4, 3, or 2 minutes, or 60, 45, 30, 20, 10 or 5 seconds before the blood flow is initiated. In some aspects, the solution is applied at least 1 minute or at least 2, 3, 4, 5 minutes before the blood flow is initiated.

In some aspects, the solution is flushed through the AVF, e.g., with a needle. In one aspect, the method further entails closing the AVF after the AVF is flushed with the solution.

In some aspects, in addition to the application of the solution as described above, or alternatively, the solution is injected into an enclosed lumen generated by clamping the proximal vein and artery of an established AVF.

In one aspect, the solution is applied within about 5 minutes (or alternatively within 10, 9, 8, 7, 6, 4, 3, or 2 minutes) following vein dilation or rubbing of the vein portion of the AVF, which is used to enlarge the internal diameter of the vein. Applying the solution to the mechanically dilated or rubbed surface of the vein interior can reduce loss of the synthetic proteoglycan on the surface during rubbing.

In certain embodiments, the disclosure provides a method for establishing a vascular access in a patient, comprising applying a solution to a wall of a blood vessel in a vascular access; and restoring or initiating blood flow in the vascular access, wherein the solution comprises an effective amount of a synthetic proteoglycan comprising a glycan having from about 1 to about 80 collagen-binding peptide(s) bonded to the glycan.

In certain embodiments, the disclosure provides a method for establishing a vascular access in a patient, comprising applying a solution to a wall of a blood vessel in a vascular access; and restoring or initiating blood flow in the vascular access, wherein the solution comprises an effective amount of a synthetic proteoglycan comprising a glycan having from about 5 to about 40 collagen-binding peptide(s) bonded to the glycan.

In certain embodiments, the disclosure provides a method for establishing a vascular access in a patient, comprising applying a solution to a wall of a blood vessel in a vascular access; and restoring or initiating blood flow in the vascular access, wherein the solution comprises an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a method for establishing a vascular access in a patient, comprising applying a solution to a wall of a blood vessel in a vascular access; and restoring or initiating blood flow in the vascular access, wherein the solution comprises an effective amount of a synthetic proteoglycan comprising a glycan having from about 5 to about 40 collagen-binding peptide(s) bonded to the glycan, wherein the glycan comprises dermatan sulfate and the collagen-binding peptide(s) comprises an amino acid sequence of RRANAALKAGELYKSILY (SEQ ID NO: 1).

In certain embodiments, the disclosure provides a method for establishing a vascular access in a patient, comprising applying a solution to a wall of a blood vessel in a vascular access; and restoring or initiating blood flow in the vascular access, wherein the solution comprises an effective amount of a synthetic proteoglycan comprising dermatan sulfate and collagen-binding peptide(s) comprising an amino acid sequence of RRANAALKAGELYKSILY (SEQ ID NO: 1).

In certain embodiments, the disclosure provides a method for improving maturation of an arteriovenous fistula (AVF) in a patient in need of hemodialysis, comprising applying a solution to the internal wall of a lumen of an AVF; and restoring or initiating blood flow in the AVF, wherein the solution comprises an effective amount of a synthetic proteoglycan comprising a glycan having from about 1 to about 80 collagen-binding peptide(s) bonded to the glycan.

In certain embodiments, the disclosure provides a method for improving maturation of an arteriovenous fistula (AVF) in a patient in need of hemodialysis, comprising applying a solution to the internal wall of a lumen of an AVF; and restoring or initiating blood flow in the AVF, wherein the solution comprises an effective amount of a synthetic proteoglycan comprising a glycan having from about 5 to about 40 collagen-binding peptide(s) bonded to the glycan.

In certain embodiments, the disclosure provides a method for improving maturation of an arteriovenous fistula (AVF) in a patient in need of hemodialysis, comprising applying a solution to the internal wall of a lumen of an AVF; and restoring or initiating blood flow in the AVF, wherein the solution comprises an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a method for improving maturation of an arteriovenous fistula (AVF) in a patient in need of hemodialysis, comprising applying a solution to the internal wall of a lumen of an AVF; and restoring or initiating blood flow in the AVF, wherein the solution comprises an effective amount of a synthetic proteoglycan comprising a glycan having from about 5 to about 40 collagen-binding peptide(s) bonded to the glycan, wherein the glycan comprises dermatan sulfate and the collagen-binding peptide(s) comprises an amino acid sequence of RRANAALKAGELYKSILY (SEQ ID NO: 1).

In certain embodiments, the disclosure provides a method for improving maturation of an arteriovenous fistula (AVF) in a patient in need of hemodialysis, comprising applying a solution to the internal wall of a lumen of an AVF; and restoring or initiating blood flow in the AVF, wherein the solution comprises an effective amount of a synthetic proteoglycan comprising dermatan sulfate and the collagen-binding peptide(s) comprising an amino acid sequence of RRANAALKAGELYKSILY (SEQ ID NO: 1).

It is contemplated that the synthetic proteoglycans provided in the solution can be tailored with respect to the peptide identity, the number of peptides attached to the glycosaminoglycan (GAG) backbone, and the GAG backbone identity to promote maturation of AVF. Thus, a number of molecular design parameters can be engineered to optimize the target effect.

It is contemplated that the synthetic proteoglycans provided in the solution can be tailored with respect to the peptide identity, the number of peptides attached to the glycosaminoglycan (GAG) backbone, and the GAG backbone identity to promote maturation of AVF or to reduce graft failure.

Another embodiment of the present disclosure provides methods and associated compositions for improving the success rate and/or reducing failure of a surgical bypass procedure. Bypass grafts are used as one form of treatment of arterial blockage in both coronary artery disease (CAD) and peripheral artery disease (PAD). Approximately 500,000 coronary artery bypass graft (CABG) procedures and over 70,000 peripheral bypass graft procedures are performed annually in the US. Most commonly, an autologous vessel graft is harvested, often from the saphenous vein.

Despite the prevalence of surgical bypass with autologous vein grafts to restore blood flow, there are a large number of vein graft failures (VGF) in both CAD and PAD. In the periphery alone, vein graft failure rates reach levels of 50% failure within 5 years. While 5% to 10% of vein grafts fail shortly after implantation due to technical factors and acute thrombosis, mid-term failure (3 to 24 months) may occur in another 20% to 30% of cases and can lead to costly surveillance, reintervention procedures and amputation. The 12-month incidence of vein graft failure in CLI patients (n=1219) was 29% during a two-decade experience at the Brigham and Women's Hospital. The consequences of vein graft failure are often severe for the patient, including recurrent ischemic symptoms, debilitating surgery and limb loss. To date, pharmacotherapies and technical innovations have had little impact on reducing vein graft failure.

It is contemplated that injuries to the fragile endothelial layer of vein graft conduits, whether caused by vein graft harvesting, preservation media, excessive manipulation in preparation for bypass, or ischemia and reperfusion injury, result in a platelet mediated inflammatory response within the vessel wall after implantation. Such endothelial injuries and ECM-platelet activation cascade can result in early VGF via acute inflammation and thrombosis, or delayed VGF via neointimal hyperplasia. Limiting the exposure of the vein graft sub-endothelial matrix to circulating platelets after implantation, therefore, can help reduce acute vessel wall inflammation, improve re-epithelialization and limit excessive neointimal hyperplasia that may lead to vessel occlusion and VGF.

In accordance with one embodiment of the present disclosure, therefore, provided is a method for preparing a vascular graft (e.g., a vein graft) by contacting the internal wall of a section of a blood vessel with a solution that contains a synthetic proteoglycan of the disclosure. One way of implementing the contact is to soak the section in the solution. Conditions for this contact can vary but can be readily determined, depending on the concentration of the synthetic proteoglycan and the characteristics of the blood vessel, such that there is a suitable amount of the synthetic proteoglycan bound to the internal wall. The vascular graft prepared with such a method is also within the scope of the present disclosure.

Once the graft is prepared, it can be implanted to a patient in need thereof. The surgical bypass procedure can be readily carried out by a medical professional. Once implanted, the synthetic proteoglycan bound to the internal wall of the grant can help reduce acute vessel wall inflammation, improve re-epithelialization of the graft and limit excessive neointimal hyperplasia of the graft, resulting in reduced graft failure.

In one embodiment, whether the graft has been treated with a synthetic proteoglycan as described above, during or following the bypass procedure, a solution of the synthetic proteoglycan can be injected into the lumen of the graft such that the synthetic proteoglycan will bind to the internal wall of the graft. In one aspect, the injection is done before blood flow is restored or started through the graft. In another aspect, the injection is done shortly after (e.g., within 10 minutes, within 5 minutes, or within 1 minute) the blood flow is restored or started.

In certain embodiments, the disclosure provides a method for preparing a vascular graft for a bypass surgery, comprising contacting the internal wall of a section of a blood vessel with a solution comprising an effective amount of a synthetic proteoglycan comprising a glycan having from about 1 to about 80 collagen-binding peptide(s) bonded to the glycan.

In certain embodiments, the disclosure provides a method for preparing a vascular graft for a bypass surgery, comprising contacting the internal wall of a section of a blood vessel with a solution comprising an effective amount of a synthetic proteoglycan comprising a glycan having from about 5 to about 40 collagen-binding peptide(s) bonded to the glycan.

In certain embodiments, the disclosure provides a method for preparing a vascular graft for a bypass surgery, comprising contacting the internal wall of a section of a blood vessel with a solution comprising an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a method for preparing a vascular graft for a bypass surgery, comprising contacting the internal wall of a section of a blood vessel with a solution comprising an effective amount of a synthetic proteoglycan comprising a glycan having from about 5 to about 40 collagen-binding peptide(s) bonded to the glycan, wherein the glycan comprises dermatan sulfate and the collagen-binding peptide(s) comprises an amino acid sequence of RRANAALKAGELYKSILY (SEQ ID NO: 1).

In certain embodiments, the disclosure provides a method for preparing a vascular graft for a bypass surgery, comprising contacting the internal wall of a section of a blood vessel with a solution comprising an effective amount of a synthetic proteoglycan comprising dermatan sulfate and collagen-binding peptide(s) comprising an amino acid sequence of RRANAALKAGELYKSILY (SEQ ID NO: 1).

In certain embodiments, the disclosure provides a vascular graft comprising a section of a blood vessel comprising an internal wall bound to an effective amount of a synthetic proteoglycan comprising a glycan having from about 1 to about 80 collagen-binding peptide(s) bonded to the glycan.

In certain embodiments, the disclosure provides a vascular graft comprising a section of a blood vessel comprising an internal wall bound to an effective amount of a synthetic proteoglycan comprising a glycan having from about 5 to about 40 collagen-binding peptide(s) bonded to the glycan.

In certain embodiments, the disclosure provides a vascular graft comprising a section of a blood vessel comprising an internal wall bound to an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a vascular graft comprising a section of a blood vessel comprising an internal wall bound to an effective amount of a synthetic proteoglycan comprising a glycan having from about 5 to about 40 collagen-binding peptide(s) bonded to the glycan, wherein the glycan comprises dermatan sulfate, and collagen-binding peptide(s) comprising an amino acid sequence of RRANAALKAGELYKSILY (SEQ ID NO: 1).

In certain embodiments, the disclosure provides a vascular graft comprising a section of a blood vessel comprising an internal wall bound to an effective amount of a synthetic proteoglycan comprising dermatan sulfate and collagen-binding peptide(s) comprising an amino acid sequence of RRANAALKAGELYKSILY (SEQ ID NO: 1).

In certain embodiments, the disclosure provides a method for preventing or reducing graft failure in a patient undergoing a bypass grafting procedure, comprising implanting a graft into the circulation system of the patient, wherein the graft comprises an internal wall bound to an effective amount of a synthetic proteoglycan comprising a glycan having from about 1 to about 80 collagen-binding peptide(s) bonded to the glycan.

In certain embodiments, the disclosure provides a method for preventing or reducing graft failure in a patient undergoing a bypass grafting procedure, comprising implanting a graft into the circulation system of the patient, wherein the graft comprises an internal wall bound to an effective amount of a synthetic proteoglycan comprising a glycan having from about 5 to about 40 collagen-binding peptide(s) bonded to the glycan.

In certain embodiments, the disclosure provides a method for preventing or reducing graft failure in a patient undergoing a bypass grafting procedure, comprising implanting a graft into the circulation system of the patient, wherein the graft comprises an internal wall bound to an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a method for preventing or reducing graft failure in a patient undergoing a bypass grafting procedure, comprising implanting a graft into the circulation system of the patient, wherein the graft comprises an internal wall bound to an effective amount of a synthetic proteoglycan comprising a glycan having from about 5 to about 40 collagen-binding peptide(s) bonded to the glycan, wherein the glycan comprises dermatan sulfate, and collagen-binding peptide(s) comprises an amino acid sequence of RRANAALKAGELYKSILY (SEQ ID NO: 1).

In certain embodiments, the disclosure provides a method for preventing or reducing graft failure in a patient undergoing a bypass grafting procedure, comprising implanting a graft into the circulation system of the patient, wherein the graft comprises an internal wall bound to an effective amount of a synthetic proteoglycan comprising dermatan sulfate and the collagen-binding peptide(s) comprising an amino acid sequence of RRANAALKAGELYKSILY (SEQ ID NO: 1).

In certain embodiments, the disclosure provides a method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising a glycan having from about 1 to about 80 collagen-binding peptide(s) bonded to the glycan.

In certain embodiments, the disclosure provides a method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising a glycan having from about 5 to about 40 collagen-binding peptide(s) bonded to the glycan.

In certain embodiments, the disclosure provides a method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising a glycan having from about 5 to about 40 collagen-binding peptide(s) bonded to the glycan, wherein the glycan comprises dermatan sulfate.

In certain embodiments, the disclosure provides a method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising a glycan having from about 5 to about 40 collagen-binding peptide(s) bonded to the glycan, wherein the glycan comprises dermatan sulfate and collagen-binding peptide(s) comprising an amino acid sequence of RRANAALKAGELYKSILY (SEQ ID NO: 1).

In certain embodiments, the disclosure provides a method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising dermatan sulfate and the collagen-binding peptide(s) comprising an amino acid sequence of RRANAALKAGELYKSILY (SEQ ID NO: 1).

In one embodiment, the molecule configuration consists of a dermatan sulfate (DS) GAG backbone with attached collagen-binding peptide(s). DS may be useful in AVF applications because of its ability to promote epithelial cell migration and proliferation.

It is contemplated that other variants of GAG-peptide provided herein are also capable of inhibiting platelet activation through binding to type I collagen. In one embodiment the synthetic proteoglycans include a collagen-binding peptide such as RRANAALKAGELYKSILY (SEQ ID NO: 1), referred to as “SILY”.

In another embodiment, the synthetic proteoglycan comprises collagen-binding peptide(s) (SILY) conjugated to GAG backbones comprising heparin (Hep-SILY), dermatan sulfate (DS-SILY), or dextran (Dex-SILY) (see, e.g., US 2011/0020298 and 2013/0190246).

DS-SILY refers to the synthetic proteoglycan having about 5-20 SILY peptide(s) conjugated to dermatan sulfate (DS). In some embodiments, the synthetic proteoglycan comprises 5-10 SILY pepetide(s) or 10-15 SILY pepetide(s) or 5-20 SILY peptide(S) conjugated to dermatan sulfate. In some embodiments, the synthetic proteoglycan comprises from about 1 to about 75 percent (%) functionalization, or from about 5 to about 30 percent (%) functionalization, wherein the percent (%) functionalization is determined by a percent of disaccharide units on the dermatan sulfate which are functionalized with SILY peptide(s). DS-SILY optionally contains a linker between the SILY peptide(s) and DS.

### 3. Synthetic Proteoglycans

In one embodiment, the synthetic proteoglycan comprises a glycan having from about 1 to about 80 collagen-binding peptide(s) bonded to the glycan.

In various embodiments of the methods disclosed herein, the glycan is dextran, chondroitin, chondroitin sulfate, dermatan, dermatan sulfate, heparan sulfate, heparin, keratin, keratan sulfate, or hyaluronic acid. In some embodiments the glycan can be any glycan (e.g., glycosaminoglycan or polysaccharide). In some embodiments, the glycan is dextran. In some embodiments, the glycan is chondroitin. In other embodiments, the glycan is chondroitin sulfate. In some embodiments, the glycan is dermatan. In some embodiments, the glycan is dermatan sulfate. In other embodiments, the glycan is heparan sulfate. In other embodiments, the glycan is heparin. In other embodiments, the glycan is keratin. In some embodiments, the glycan is keratan sulfate. In other embodiments, the glycan is hyaluronic acid. Various glycans may be employed including, a wide range of molecular weights, such as from about 1 kDa to about 2 MDa, or from about 10 kDa to about 2 MDa. In some embodiments, the glycan is from about 3 to about 5 MDa. In some embodiments, the glycan is up to about 3 MDa, or up to about 5 MDa, or up to about 60 MDa.

The peptide(s) can be bonded to the glycan directly or via a linker. In some embodiments, the linker can be any suitable bifunctional linker, e.g., N-[β-maleimidopropionic acid]hydrazide (BMPH), 3-(2-pyridyldithio)propionyl hydrazide (PDPH), and the like. In any of the various embodiments described herein, the sequence of the peptide may be modified to include a glycine-cysteine (GC) attached to the C-terminus of the peptide and/or a glycine-cysteine-glycine (GCG) attached to the N-terminus to provide an attachment point for a glycan or a glycan-linker conjugate. In certain embodiments, the linker is N-[β-maleimidopropionic acid]hydrazide (BMPH). In certain embodiments, the linker is 3-(2-pyridyldithio)propionyl hydrazide (PDPH). In some embodiments, the peptide to linker ratio is from about 1:1 to about 5:1. In other embodiments, the peptide to linker ratio is from about 1:1 to about 10:1. In other embodiments, the peptide to linker ratio is from about 1:1 to about 2:1, or from about 1:1 to about 3:1, or from about 1:1 to about 4:1, or from about 1:1 to about 5:1, or from about 1:1 to about 6:1, or from about 1:1 to about 7:1, or from about 1:1 to about 8:1, or from about 1:1 to about 9:1. In one embodiment, the peptide linker ratio is about 1:1. In one embodiment, the peptide linker ratio is about 2:1. In one embodiment, the peptide linker ratio is about 3:1. In one embodiment, the peptide linker ratio is about 4:1. In one embodiment, the peptide linker ratio is about 5:1. In one embodiment, the peptide linker ratio is about 6:1. In one embodiment, the peptide linker ratio is about 7:1. In one embodiment, the peptide linker ratio is about 8:1. In one embodiment, the peptide linker ratio is about 9:1. In one embodiment, the peptide linker ratio is about 10:1.

Depending on the desired properties of the synthetic proteoglycan, the total number of peptides bonded to the glycan can be varied. In certain embodiments, the total number of peptides present in the synthetic proteoglycan is from about 1 or 2 to about 160, or from about 10 to about 160, or from about 20 to about 160, or from about 30 to about 160, or from about 40 to about 160, or from about 40 to about 150, or from about 40 to about 140, or from about 10 to about 120, or from about 20 to about 110, or from about 20 to about 100, or from about 20 to about 90, or from about 30 to about 90, or from about 40 to about 90, or from about 50 to about 90, or from about 50 to about 80, or from about 60 to about 80, or about 10, or about 20, or about 30, or about 40, or about 50, or about 60, or about 70, or about 80, or about 90, or about 100, or about 110, or about 120. In certain embodiments, the synthetic proteoglycan comprises less than about 50 peptides. In various embodiments, the synthetic proteoglycan comprises from about 5 to about 40 peptides. In some embodiments, the synthetic proteoglycan comprises from about 10 to about 40 peptides. In other embodiments, the synthetic proteoglycan comprises from about 5 to about 20 peptides. In various embodiments, the synthetic proteoglycan comprises from about 4 to about 18 peptides. In certain embodiments, the synthetic proteoglycan comprises less than about 20 peptides. In certain embodiments, the synthetic proteoglycan comprises less than about 18 peptides. In certain embodiments, the synthetic proteoglycan comprises less than about 15 peptides. In certain embodiments, the synthetic proteoglycan comprises less than about 10 peptides. In certain embodiments, the synthetic proteoglycan comprises about 20 peptides. In certain embodiments, the synthetic proteoglycan comprises about 40 peptides. In certain embodiments, the synthetic proteoglycan comprises about 18 peptides. In certain embodiments, the synthetic proteoglycan comprises from about 5 to about 40, or from about 10 to about 40, or from about 5 to about 20, or from about 4 to about 18, or about 10, or about 11, or about 18, or about 20 peptides.

In any of the embodiments described herein, the number of peptides per glycan is an average, where certain synthetic proteoglycans in a composition may have more peptides per glycan and certain synthetic proteoglycans have less peptides per glycan. Accordingly, in certain embodiments, the number of peptides as described herein is an average in a composition of synthetic proteoglycans. For example, in certain embodiments, the synthetic proteoglycans are a composition where the average number of peptides per glycan is about 5. In other embodiments, the average number of peptides per glycan is about 6, or about 7, or about 8, or about 9, or about 10, or about 11, or about 12, or about 13, or about 14, or about 15, or about 16, or about 17, or about 18, or about 19, or about 20, or about 25, or about 30. In certain embodiments, the number of peptides per glycan may be described as a “percent (%) functionalization” based on the percent of disaccharide units which are functionalized with peptide on the glycan backbone. For example, the total number of available disaccharide units present on the glycan can be calculated by dividing the molecular weight (or the average molecular weight) of a single disaccharide unit (e.g., about 550-800 Da, or from about 650-750 Da) by the molecular weight of the glycan (e.g., about 25 kDa up to about 70 kDa, or even about 100 kDa). For example, in some embodiments, the number of available disaccharide units present on the glycan is from about 10 to about 80, or from about 10 to about 70, or from about 15 to about 70, or from about 20 to about 70, or from about 30 to about 70, or from about 35 to about 70, or from about 40 to about 70, or from about 10 to about 50, or from about 20 to about 50, or from about 25 to about 50, or from about 10 to about 30, or from about 15 to about 30, or from about 20 to about 30, or about 15, or about 20, or about 25, or about 30, or about 35, or about 40, or about 45, or about 50, or about 55, or about 60, or about 65, or about 70.

Therefore, in certain embodiments, the glycan comprises from about 1 to about 50, or from about 5 to about 30% functionalization, or about 25% functionalization, wherein the percent (%) functionalization is determined by a percent of disaccharide units on the glycan which are functionalized with peptide. In some embodiments, the percent (%) functionalization of the glycan is from about 1% to about 50%, or from about 3% to about 40%, or from about 5% to about 30%, or from about 10% to about 20%, or about 1%, or about 2%, or about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%.

In one aspect, the collagen-binding peptide has binding affinity to one or more of collagen types I, II, III, or IV. In some embodiments, the collagen-binding peptide binds to type I collagen. In other embodiments, the collagen-binding peptide binds to type IV collagen. In certain embodiments, one or more collagen-binding peptide having a specified binding affinity can be used in the synthetic proteoglycans described herein. For example, the synthetic proteoglycans can comprise at least one collagen-binding peptide which has binding affinity to type I collagen and at least one collagen-binding peptide which has binding affinity to type IV collagen. In another aspect, the collagen-binding peptides have binding affinity to type I collagen. In another aspect, the collagen-binding peptides have binding affinity to type IV collagen. In certain aspects, the collagen-binding peptides have binding affinity to type II collagen. In certain aspects, the collagen-binding peptides have binding affinity to type III collagen.

Suitable collagen-binding peptides are known (see, e.g., US 2013/0190246, US 2012/0100106, and US 2011/0020298, the disclosures of which are incorporated herein by reference in their entirety) or can be found by methods known in the art. In certain embodiments, the collagen-binding peptide comprises from about 5 to about 40 amino acids. In some embodiments, these peptides have homology to the amino acid sequence of a small leucine-rich proteoglycan, a platelet receptor sequence, or a protein that regulates collagen fibrillogenesis.

In various embodiments, the collagen-binding peptide comprises an amino acid sequence selected from:

i)
(SEQ ID NO: 1)
RRANAALKAGELYKSILY,
(SEQ ID NO: 2)
RLDGNEIKR,
(SEQ ID NO: 3)
AHEEISTTNEGVM,
(SEQ ID NO: 4)
GELYKSILY,
(SEQ ID NO: 5)
NGVFKYRPRYFLYKHAYFYPPLKRFPVQ,
(SEQ ID NO: 6)
CQDSETRTFY,
(SEQ ID NO: 7)
TKKTLRT,
(SEQ ID NO: 8)
GLRSKSKKFRRPDIQYPDATDEDITSHM,
(SEQ ID NO: 9)
SQNPVQP,
(SEQ ID NO: 10)
SYIRIADTNIT,
(SEQ ID NO: 11)
KELNLVYT,
(SEQ ID NO: 12)
GSITTIDVPWNVGC,
(SEQ ID NO: 13)
GSITTIDVPWNV;
(SEQ ID NO: 14)
RRANAALKAGELYKCILY,
(SEQ ID NO: 15)
GELYKCILY,
(SEQ ID NO: 16)
GQLYKSILY,
or
(SEQ ID NO: 17)
RRANAALKAGQLYKSILY

or ii) a peptide comprising a sequence with at least about 80% sequence identity to the amino acid sequence of i) and capable of binding to collagen.

In certain embodiments, the collagen-binding peptide(s) is RRANAALKAGELYKSILY (SEQ ID NO: 1) or a peptide having at least about 80% sequence to RRANAALKAGELYKSILY (SEQ ID NO: 1) and capable of binding to collagen. In some embodiments, the peptide sequence comprises a sequence with at least about 80% sequence identity, or at least about 83% sequence identity, or at least about 85% sequence identity, or at least about 90% sequence identity, or at least about 95% sequence identity, or at least about 98% sequence identity to the amino acid sequence of i) and capable of binding to collagen. In certain embodiments, the collagen-binding peptide is at least about 80%, or at least about 83%, or at least about 85%, or at least about 90%, or at least about 95%, or at least about 98%, or at least about 100% homologous with the collagen-binding unit(s) of the von Willebrand factor or a platelet collagen receptor as described in Chiang, T. M. et al. J. Biol. Chem., 2002, 277: 34896-34901; Huizinga, E. G. et al., Structure, 1997, 5: 1147-1156; Romijn, R. A. et al., J. Biol. Chem., 2003, 278: 15035-15039; and Chiang, et al., Cardio. & Haemato. Disorders-Drug Targets, 2007, 7: 71-75, each incorporated herein by reference.

In various embodiments, the collagen-binding peptide comprises an amino acid spacer. Accordingly, in certain embodiments, the collagen-binding peptide comprises an amino acid sequence selected from:

i)
(SEQ ID NO: 64)
RRANAALKAGELYKSILYGC,
(SEQ ID NO: 66)
RLDGNEIKRGC,
(SEQ ID NO: 67)
AHEEISTTNEGVMGC,
(SEQ ID NO: 68)
GCGGELYKSILY,
(SEQ ID NO: 69)
NGVFKYRPRYFLYKHAYFYPPLKRFPVQGC,
(SEQ ID NO: 70)
CQDSETRTFYGC,
(SEQ ID NO: 71)
TKKTLRTGC,
(SEQ ID NO: 72)
GLRSKSKKFRRPDIQYPDATDEDITSHMGC,
(SEQ ID NO: 73)
SQNPVQPGC,
(SEQ ID NO: 74)
SYIRIADTNITGC,
(SEQ ID NO: 75)
KELNLVYTGC,
(SEQ ID NO: 12)
GSITTIDVPWNVGC,
(SEQ ID NO: 76)
GCGGELYKSILYGC
or
(SEQ ID NO: 65)
GELYKSILYGC;

or

ii) a peptide comprising a sequence with at least about 80% sequence identity to the amino acid sequence of i) and capable of binding to collagen. In some embodiments, the peptide sequence comprises a sequence with at least about 80% sequence identity, or at least about 83% sequence identity, or at least about 85% sequence identity, or at least about 90% sequence identity, or at least about 95% sequence identity, or at least about 98% sequence identity to the amino acid sequence of i) and capable of binding to collagen.

In one embodiment, the synthetic proteoglycan comprises dermatan sulfate having from about 5 to about 40 collagen-binding peptide(s) bonded thereto and wherein the collagen-binding peptide(s) is RRANAALKAGELYKSILY (SEQ ID NO: 1) or a peptide having at least about 80% sequence identity to RRANAALKAGELYKSILY (SEQ ID NO: 1) and capable of binding to collagen.

Similarly for a collagen-binding peptide, a synthetic peptide derived from a phage display library selected for collagen binding can be generated. The peptide can be synthesized and evaluated for binding to collagen by any of the techniques such as SPR, ELISA, ITC, affinity chromatography, or others known in the art. An example could be a biotin modified peptide sequence (e.g., SILYbiotin) that is incubated on a microplate containing immobilized collagen. A dose response binding curve can be generated using a streptavidin-chromophore to determine the ability of the peptide to bind to collagen.

In various embodiments described herein, the peptides described herein can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts to the side chain of the amino acid that has been replaced. Non-conservative substitutions may too be possible, provided that they do not substantially affect the binding activity of the peptide (i.e., collagen-binding affinity).

### 4. Synthesis of Synthetic Proteoglycans

The peptides used in the method described herein (i.e., the collagen-binding peptide) may be purchased from a commercial source or partially or fully synthesized using methods well known in the art (e.g., chemical and/or biotechnological methods). In certain embodiments, the peptides are synthesized according to solid phase peptide synthesis protocols that are well known in the art. In another embodiment, the peptide is synthesized on a solid support according to the well-known Fmoc protocol, cleaved from the support with trifluoroacetic acid and purified by chromatography according to methods known to persons skilled in the art. In other embodiments, the peptide is synthesized utilizing the methods of biotechnology that are well known to persons skilled in the art. In one embodiment, a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression, and the peptide is then isolated from the host organism or the growth medium, e.g., by affinity purification. Recombinant DNA technology methods are described in Sambrook et al., “Molecular Cloning: A Laboratory Manual”, 3rd Edition, Cold Spring Harbor Laboratory Press, (2001), incorporated herein by reference, and are well-known to the skilled artisan.

In certain embodiments, the peptides are covalently bonded to the glycan directly (i.e., without a linker). In such embodiments, the synthetic proteoglycan may be formed by covalently bonding the peptides to the glycan through the formation of one or more amide, ester or imino bonds between an acid, aldehyde, hydroxy, amino, or hydrazo group on the glycan. All of these methods are known in the art. See, e.g., Hermanson G. T., Bioconjugate Techniques, Academic Press, pp. 169-186 (1996), incorporated herein by reference. As shown in Scheme 1, the glycan (e.g., chondroitin sulfate “CS”) can be oxidized using a periodate reagent, such as sodium periodate, to provide aldehyde functional groups on the glycan (e.g., “ox-CS”) for covalently bonding the peptides to the glycan. In such embodiments, the peptides may be covalently bonded to a glycan by reacting a free amino group of the peptide with an aldehyde functional groups of the oxidized glycan, e.g., in the presence of a reducing agent, utilizing methods known in the art.

In embodiments where the peptides are covalently bonded to the glycan via a linker, the oxidized glycan (e.g., “ox-CS”) can be reacted with a linker (e.g., any suitable bifunctional linker, such as 3-(2-pyridyldithio)propionyl hydrazide (PDPH) or N-[β-maleimidopropionic acid]hydrazide (BMPH)) prior to contacting with the peptides. The linker typically comprises about 1 to about 30 carbon atoms, or about 2 to about 20 carbon atoms. Lower molecular weight linkers (i.e., those having an approximate molecular weight of about 20 to about 500) are typically employed. In addition, structural modifications of the linker are contemplated. For example, amino acids may be included in the linker, including but not limited to, naturally occurring amino acids as well as those available from conventional synthetic methods, such as beta, gamma, and longer chain amino acids.

As shown in Scheme 1, in one embodiment, the peptides are covalently bonded to the glycan (e.g., chondroitin sulfate “CS”) by reacting an aldehyde function of the oxidized glycan (e.g., “ox-CS”) with N-[β-maleimidopropionic acid]hydrazide (BMPH) to form an glycan intermediate (e.g., “BMPH-CS”) and further reacting the glycan intermediate with peptides containing at least one free thiol group (i.e., —SH group) to yield the synthetic proteoglycan. In yet another embodiment, the sequence of the peptides may be modified to include an amino acid residue or residues that act as a spacer between the HA- or Collagen-binding peptide sequence and a terminating cysteine (C). For example a glycine-cysteine (GC) or a glycine-glycine-glycine-cysteine (GGGC) or glycine-serine-glycine-cysteine (GSGC) segment may be added to provide an attachment point for the glycan intermediate.

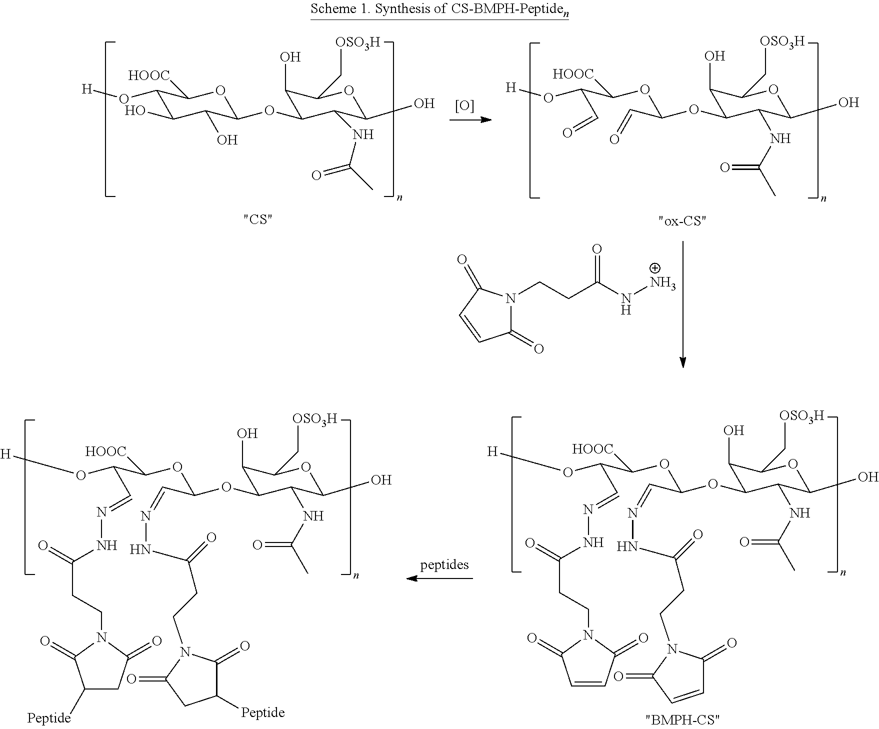

### 5. Compositions

In one embodiment, the synthetic proteoglycan is administered in a composition. The present disclosure provides compositions comprising a synthetic proteoglycan and a pharmaceutically acceptable carrier. Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into an AVF. Diluent or carriers employed in the compositions can be selected so that they do not diminish the desired effects of the synthetic proteoglycan. Examples of suitable compositions include aqueous solutions, for example, a solution in isotonic saline, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents.

In certain embodiments, the composition is an aqueous solution. Aqueous solutions are suitable for use in composition formulations based on ease of formulation, as well as an ability to easily administer such compositions by means of instilling the solution in. In certain embodiments, the compositions are suspensions, viscous or semi-viscous gels, or other types of solid or semi-solid compositions. In some embodiments, the composition is in the form of foams, ointments, liquid wash, gels, sprays and liposomes, which are very well known in the art. Alternatively, the topical administration is an infusion of the provided synthetic proteoglycan to the AVF via a device selected from a pump-catheter system, a continuous or selective release device, or an adhesion barrier. In certain embodiments, the composition is a solution that is directly applied to or contacts the internal wall of a vein or artery of the AVF. In some embodiments, the composition comprises a polymer matrix. In other embodiments, the composition is absorbable. In certain embodiments, the composition comprises a pH buffering agent. In some embodiments, the composition contains a lubricity-enhancing agent.

In certain embodiments, a polymer matrix or polymeric material is employed as a pharmaceutically acceptable carrier or support for the anti-adhesion composition. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, the anti-adhesion compositions provided herein is formulated as films, gels, foams, or and other dosage forms.

Suitable ionic strength modifying agents include, for example, glycerin, propylene glycol, mannitol, glucose, dextrose, sorbitol, sodium chloride, potassium chloride, and other electrolytes.

In certain embodiments, the solubility of the synthetic proteoglycan may need to be enhanced. In such cases, the solubility may be increased by the use of appropriate formulation techniques, such as the incorporation of solubility-enhancing compositions such as mannitol, ethanol, glycerin, polyethylene glycols, propylene glycol, poloxomers, and others known in the art.

In certain embodiments, the composition contains a lubricity-enhancing agent. As used herein, lubricity-enhancing agents refer to one or more pharmaceutically acceptable polymeric materials capable of modifying the viscosity of the pharmaceutically acceptable carrier. Suitable polymeric materials include, but are not limited to: ionic and non-ionic water soluble polymers; hyaluronic acid and its salts, chondroitin sulfate and its salts, dextrans, gelatin, chitosans, gellans, other proteoglycans or polysaccharides, or any combination thereof; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); gums such as tragacanth and xanthan gum; gellan gums; alginate and sodium alginate; chitosans; vinyl polymers; hydrophilic polymers such as polyethylene oxides, polyoxyethylene-polyoxypropylene copolymers, and polyvinylalcohol; carboxyvinyl polymers or crosslinked acrylic acid polymers such as the “carbomer” family of polymers, e.g., carboxypolyalkylenes that may be obtained commercially under the Carbopol™ trademark; and various other viscous or viscoelastomeric substances. In one embodiment, a lubricity enhancing agent is selected from the group consisting of hyaluronic acid, dermatan, chondroitin, heparin, heparan, keratin, dextran, chitosan, alginate, agarose, gelatin, hydroxypropyl cellulose, hydroxyethyl cellulose, hydroxypropyl methylcellulose, hydroxypropyl methylcellulose phthalate, methyl cellulose, carboxymethyl cellulose, and etherified cellulose, polyvinyl alcohol, polyvinylpyrrolidinone, povidone, carbomer 941, carbomer 940, carbomer 971P, carbomer 974P, or a pharmaceutically acceptable salt thereof. In one embodiment, a lubricity-enhancing agent is applied concurrently with the synthetic proteoglycan. Alternatively, in one embodiment, a lubricity-enhancing agent is applied sequentially to the synthetic proteoglycan. In one embodiment, the lubricity-enhancing agent is chondroitin sulfate. In one embodiment, the lubricity enhancing agent is hyaluronic acid. The lubricity-enhancing agent can change the viscosity of the composition.

For further details pertaining to the structures, chemical properties and physical properties of the above lubricity-enhancing agents, see e.g., U.S. Pat. No. 5,409,904, U.S. Pat. No. 4,861,760 (gellan gums), U.S. Pat. No. 4,255,415, U.S. Pat. No. 4,271,143 (carboxyvinyl polymers), WO 94/10976 (polyvinyl alcohol), WO 99/51273 (xanthan gum), and WO 99/06023 (galactomannans). Typically, non-acidic lubricity-enhancing agents, such as a neutral or basic agent are employed in order to facilitate achieving the desired pH of the formulation.

In some embodiments, the synthetic proteoglycans can be combined with minerals, amino acids, sugars, peptides, proteins, vitamins (such as ascorbic acid), or laminin, collagen, fibronectin, hyaluronic acid, fibrin, elastin, or aggrecan, or growth factors such as epidermal growth factor, platelet-derived growth factor, transforming growth factor beta, or fibroblast growth factor, and glucocorticoids such as dexamethasone or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

Suitable pH buffering agents for use in the anti-adhesion compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES. In certain embodiments, an appropriate buffer system (e.g., sodium phosphate, sodium acetate, sodium citrate, sodium borate or boric acid) is added to the composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 4 to about pH 8. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7. In some embodiments, the composition is in the form of a film, gel, patch, or liquid solution which comprises a polymeric matrix, pH buffering agent, a lubricity enhancing agent and a synthetic proteoglycan wherein the composition optionally contains a preservative; and wherein the pH of said composition is within the range of about pH 4 to about pH 8.

Surfactants are employed in the composition to deliver higher concentrations of synthetic proteoglycan. The surfactants function to solubilize the inhibitor and stabilize colloid dispersion, such as micellar solution, microemulsion, emulsion and suspension. Suitable surfactants comprise c polysorbate, poloxamer, polyoxyl 40 stearate, polyoxyl castor oil, tyloxapol, triton, and sorbitan monolaurate. In one embodiment, the surfactants have hydrophile/lipophile/balance (HLB) in the range of 12.4 to 13.2 and are acceptable for ophthalmic use, such as TritonX114 and tyloxapol.

In certain embodiments, stabilizing polymers, i.e., demulcents, are added to the composition. The stabilizing polymer should be an ionic/charged example, more specifically a polymer that carries negative charge on its surface that can exhibit a zeta-potential of (−)10-50 mV for physical stability and capable of making a dispersion in water (i.e. water soluble).

In one embodiment, the stabilizing polymer comprises a polyelectrolyte or polyectrolytes if more than one, from the family of cross-linked polyacrylates, such as carbomers and Pemulen®, specifically Carbomer 974p (polyacrylic acid), at a range of about 0.1% to about 0.5% w/w.

In one embodiment, the composition comprises an agent which increases the permeability of the synthetic proteoglycan to the extracellular matrix of blood vessels. Preferably the agent which increases the permeability is selected from benzalkonium chloride, saponins, fatty acids, polyoxyethylene fatty ethers, alkyl esters of fatty acids, pyrrolidones, polyvinylpyrrolidone, pyruvic acids, pyroglutamic acids or mixtures thereof.

The synthetic proteoglycan may be sterilized to remove unwanted contaminants including, but not limited to, endotoxins and infectious agents. Sterilization techniques which do not adversely affect the structure and biotropic properties of the synthetic proteoglycan can be used. In certain embodiments, the synthetic proteoglycan can be disinfected and/or sterilized using conventional sterilization techniques including propylene oxide or ethylene oxide treatment, sterile filtration, gas plasma sterilization, gamma radiation, electron beam, and/or sterilization with a peracid, such as peracetic acid. In one embodiment, the synthetic proteoglycan can be subjected to one or more sterilization processes. Alternatively, the synthetic proteoglycan may be wrapped in any type of container including a plastic wrap or a foil wrap, and may be further sterilized.

In some embodiments, preservatives are added to the composition to prevent microbial contamination during use. Suitable preservatives added to the anti-adhesion compositions comprise benzalkonium chloride, benzoic acid, alkyl parabens, alkyl benzoates, chlorobutanol, chlorocresol, cetyl alcohols, fatty alcohols such as hexadecyl alcohol, organometallic compounds of mercury such as acetate, phenylmercury nitrate or borate, diazolidinyl urea, diisopropyl adipate, dimethyl polysiloxane, salts of EDTA, vitamin E and its mixtures. In certain embodiments, the preservative is selected from benzalkonium chloride, chlorobutanol, benzododecinium bromide, methyl paraben, propyl paraben, phenylethyl alcohol, edentate disodium, sorbic acid, or polyquarternium-1. In certain embodiments, the ophthalmic compositions contain a preservative. In some embodiments, the preservatives are employed at a level of from about 0.001% to about 1.0% w/v. In certain embodiments, the ophthalmic compositions do not contain a preservative and are referred to as “unpreserved”. In some embodiments, the unit dose compositions are sterile, but unpreserved.

In some embodiments, separate or sequential administration of the synthetic proteoglycan and other agent is necessary to facilitate delivery of the composition into the AVF. In certain embodiments, the synthetic proteoglycan and the other agent can be administered at different dosing frequencies or intervals. For example, the synthetic proteoglycan can be administered daily, while the other agent can be administered less frequently. Additionally, as will be apparent to those skilled in the art, the synthetic proteoglycan and the other agent can be administered using the same route of administration or different routes of administration.

Any effective regimen for administering the synthetic proteoglycan can be used. For example, the synthetic proteoglycan can be administered as a single dose, or as a multiple-dose daily regimen. Further, a staggered regimen, for example, one to five days per week can be used as an alternative to daily treatment.

In various embodiments, the synthetic proteoglycan can be administered topically, such as by film, gel, patch, or liquid solution. In some of the embodiments, the compositions provided are in a buffered, sterile aqueous solution. In certain embodiments, the solutions have a viscosity of from about 1 to about 100 centipoises (cps), or from about 1 to about 200 cps, or from about 1 to about 300 cps, or from about 1 to about 400 cps. In some embodiments, the solutions have a viscosity of from about 1 to about 100 cps. In certain embodiments, the solutions have a viscosity of from about 1 to about 200 cps. In certain embodiments, the solutions have a viscosity of from about 1 to about 300 cps. In certain embodiments, the solutions have a viscosity of from about 1 to about 400 cps. In certain embodiments, the solution is in the form of an injectable liquid solution. In other embodiments, the compositions are formulated as viscous liquids, i.e., viscosities from several hundred to several thousand cps, gels or ointments. In these embodiments, the synthetic proteoglycan is dispersed or dissolved in an appropriate pharmaceutically acceptable carrier.

Exemplary compositions for use with the synthetic proteoglycans for catheter-based delivery may comprise: a) a synthetic proteoglycan as described herein; b) a pharmaceutically acceptable carrier; c) a polymer matrix; d) a pH buffering agent to provide a pH in the range of about pH 4 to about pH 8; and e) a water soluble lubricity enhancing agent in the concentration range of about 0.25% to about 10% total formula weight or any individual component a), b), c), d) ore), or any combinations of a), b), c), d) or e).

### 6. Pharmaceutical Formulations

Formulations contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure.

Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In making pharmaceutical compositions that include synthetic proteoglycans described herein, the active ingredient is usually diluted by an excipient or carrier and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of films, gels, patches, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin films, gels, patches, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Films used for drug delivery are well known in the art and comprise non-toxic, non-irritant polymers devoid of leachable impurities, such as polysaccharides (e.g., cellulose, maltodextrin, etc.). In some embodiments, the polymers are hydrophilic. In other embodiments, the polymers are hydrophobic. The film adheres to tissues to which it is applied, and is slowly absorbed into the body over a period of about a week. Polymers used in the thin-film dosage forms described herein are absorbable and exhibit sufficient peel, shear and tensile strengths as is well known in the art. In some embodiments, the film is injectable. In certain embodiments, the film is administered to the patient prior to, during or after surgical intervention.

Gels are used herein refer to a solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 90% water. In some embodiments, the gel described herein comprises a natural or synthetic polymeric network. In some embodiments, the gel comprises a hydrophilic polymer matrix. In other embodiments, the gel comprises a hydrophobic polymer matrix. In some embodiments, the gel possesses a degree of flexibility very similar to natural tissue. In certain embodiments, the gel is biocompatible and absorbable. In certain embodiments, the gel is administered to the patient prior to, during or after surgical intervention.

Liquid solution as used herein refers to solutions, suspensions, emulsions, drops, ointments, liquid wash, sprays, liposomes which are well known in the art. In some embodiments, the liquid solution contains an aqueous pH buffer agent which resists changes in pH when small quantities of acid or base are added. In certain embodiments, the liquid solution is administered to the patient prior to, during or after surgical intervention.

Exemplary formulations may comprise: a) synthetic proteoglycan as described herein; b) pharmaceutically acceptable carrier; c) polymer matrix; and d) pH buffering agent to provide a pH in the range of about pH 4 to about pH 8, wherein said solution has a viscosity of from about 3 to about 30 cps for a liquid solution. In certain embodiments, the solutions have a viscosity of from about 1 to about 100 centipoises (cps), or from about 1 to about 200 cps, or from about 1 to about 300 cps, or from about 1 to about 400 cps. In some embodiments, the solutions have a viscosity of from about 1 to about 100 cps. In certain embodiments, the solutions have a viscosity of from about 1 to about 200 cps. In certain embodiments, the solutions have a viscosity of from about 1 to about 300 cps. In certain embodiments, the solutions have a viscosity of from about 1 to about 400 cps.

Alternatively, exemplary formulations may comprise: a) synthetic proteoglycan as described herein; b) pharmaceutically acceptable carrier; and c) hydrophilic polymer as matrix network, wherein said compositions are formulated as viscous liquids, i.e., viscosities from several hundred to several thousand cps, gels or ointments. In these embodiments, the synthetic proteoglycan is dispersed or dissolved in an appropriate pharmaceutically acceptable carrier.

In certain embodiments, the synthetic proteoglycan, or a composition comprising the same, is lyophilized prior to, during, or after, formulation. Accordingly, also provided herein is a lyophilized composition comprising a proteoglycan or composition comprising the same as described herein.

### 7. Dosing

Suitable dosages of the synthetic proteoglycan can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.0001 mg to about 10 mg. In other illustrative aspects, effective doses ranges from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In any of the various embodiments described herein, effective doses ranges from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 mg to about 10 mg per dose. In other illustrative embodiments, effective doses can be about 1 μg, about 10 μg, about 25 μg, about 50 μg, about 75 μg, about 100 μg, about 125 μg, about 150 μg, about 200 μg, about 250 μg, about 275 μg, about 300 μg, about 350 μg, about 400 μg, about 450 μg, about 500 μg, about 550 μg, about 575 μg, about 600 μg, about 625 μg, about 650 μg, about 675 μg, about 700 μg, about 800 μg, about 900 μg, 1.0 mg, about 1.5 mg, about 2.0 mg, about 10 mg, about 100 mg, or about 100 mg to about 30 grams. In certain embodiments, the dose is from about 0.01 mL to about 10 mL.

In some embodiments, the compositions are packaged in multidose form. Preservatives are thus required to prevent microbial contamination during use. In certain embodiments, suitable preservatives as described above can be added to the compositions. In some embodiments, the composition contains a preservative. In certain embodiments the preservatives are employed at a level of from about 0.001% to about 1.0% w/v. In some embodiments, the unit dose compositions are sterile, but unpreserved.

In one embodiment, an effective amount of a composition comprising a synthetic proteoglycan and pharmaceutically acceptable carrier is administered to a patient in need to improving maturation of AVF, for instance, without limitation.

### EXAMPLES

### Example 1. Synthesis of DS-SILY

Dermatan sulfate (DS) was dissolved in 0.1 M sodium phosphate buffer at pH 5.5 to make a solution of a concentration of 20 mg/mL. The degree of functionalization was controlled by the concentration of the periodate. Periodate solutions of various concentrations were prepared by dissolving it in 0.1 M sodium phosphate buffer at pH 5.5 according to the following table.

| Target (SILY/DS) | Peridodate Concentration (mg/mL) |
|---|---|
| 20 | 2.3 |
| 10 | 1.1 |
| 5 | 0.5 |

The DS solution was mixed with the periodate solution in a ratio of 1:1 (V:V) for two hours at room temperature to provide the oxidized DS, which was purified using Biogel P6 column with phosphate buffer saline. SILY peptide having a terminal GSG-NHNH₂ bound thereto (i.e., RRANAALKAGELYKSILYGSG-NHNH₂ (SEQ ID NO: 77)) was dissolved in water to provide a concentration of 1 mg/mL using sonication if needed. The SILY peptide was slowly added to the oxidized DS at room temperature and stirred for about 2 hours protecting it from light. The pH of the reaction mixture was maintained above 6. Optionally, one mole of similarly functionalized SILY_{biotin} (biotin-labeled peptide) can be reacted with one mole of DS and then unlabeled SILY peptide can be added up (molar equivalent −1) to the number of aldehydes expected. For example, for DS-SILY₂₀, 1 mole of SILY_{biotin} and 19 moles of SILY unlabeled were added. However, the addition of SILY_{biotin} was optional. DS-SILY₂₀ was also prepared by adding 20 moles of SILY-unlabeled to one mole of DS. The product was purified with water to provide the desired DS-SILY.

### Example 2. DS-SILY was Effectively Delivered to the Fistula Vessels without Altering Standard of Care

This example demonstrates that DS-SILY, a proteoglycan described herein, could be used as a luminal vascular coating to prevent intimal hyperplasia in a native AVF. In this example, the fistulas were created in the femoral arteries and veins of pigs, either with DS-SILY treatment or with saline as a control. The results show that DS-SILY was effectively delivered to the fistula vessels without altering standard of care, and resulted in significantly less stenosis than untreated fistulas and enlarged vessel diameter.

Three delivery methods were tested in this example: (1) delivery immediately prior to blood flow, (2) delivery following intentional denudation of the fistula prior to blood flow, and (3) delivery to the formed fistula 5 minutes following restoration of blood flow.

Method 1 can be considered the primary method of delivery, and methods 2 and 3 can be used alone or in addition to method 1 in case the method 1 did not adequately result in coverage of DS-SILY to the vessel lumen. In method 2, intentional denudation of the vessel was performed by rubbing the vein with the handle of forceps, similarly to clinical application of a dilator during AVF creation. Method 3 was examined to determine if the high blood flow in the vein immediately following fistula creation resulted in damage to the endothelial cell layer that would not be addressed by initial delivery of the therapeutic. In method 3, blood flow was restored to the fistula, and then stopped by clamping the proximal vein and artery. The fistula was then flushed with DS-SILY and blood flow was again restored.

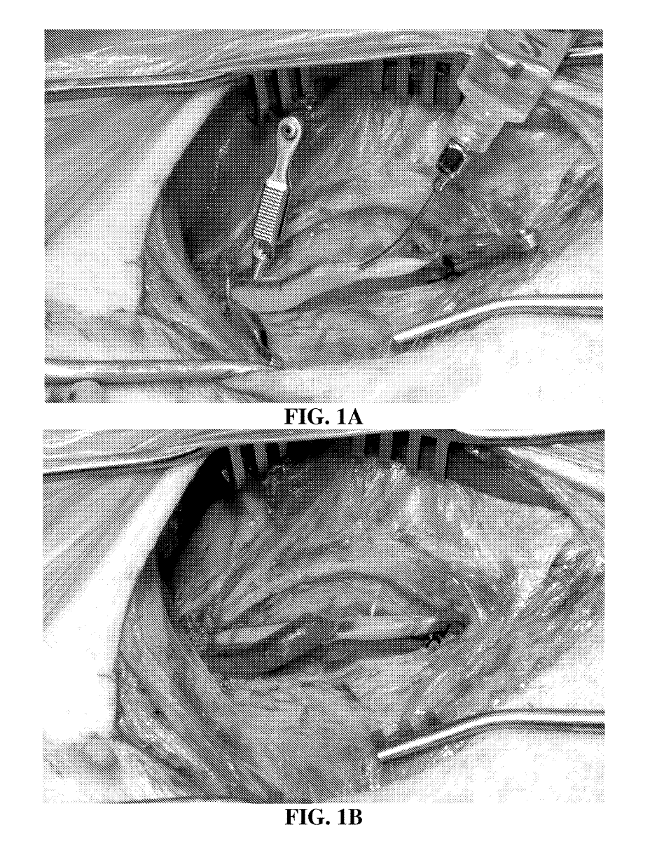

In all cases, a single suture was removed in the newly created anastomosis and the DS-SILY was flushed through the fistula using a feeding needle with a smooth ball tip. Approximately 5 mL of DS-SILY was flushed through the fistula. FIG. 1A-B show the delivery of DS-SILY to the fistula and the fistula after closure and restoration of blood flow. Animals were sacrificed approximately 2 hours following DS-SILY delivery. The fistulas were removed, rinsed in saline, and fixed with 10% formalin for 10 minutes.

Cells were stained using phalloidin. The DS-SILY molecule had a biotin tag, and was detected using a fluorescently labeled streptavidin marker. The vein was separated from the artery, and both vessels were opened so that their luminal surfaces could be examined using confocal microscopy.

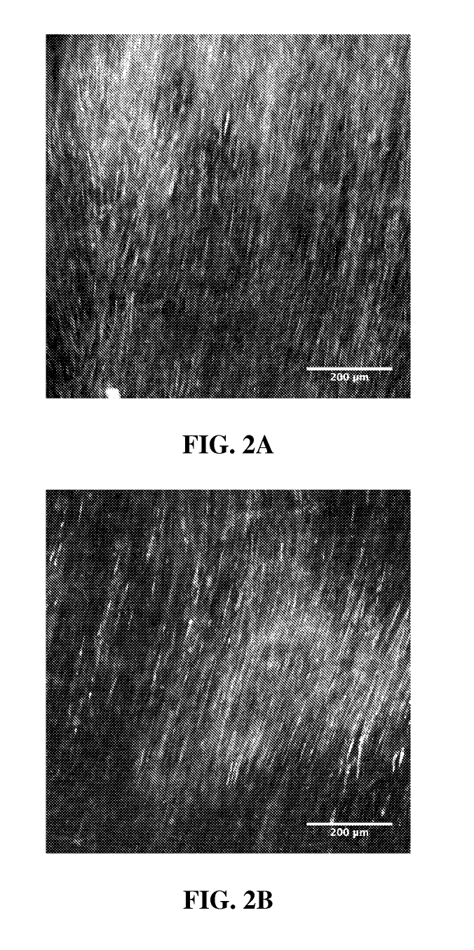

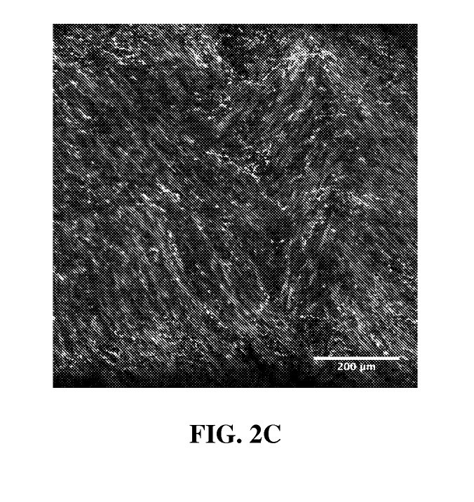

Representative images of the luminal surface of the vein 1-2 cm away from the anastamosis are shown in FIG. 2A-C. In all 3 methods, effective delivery of DS-SILY was demonstrated by the presence of the red fluorescent streptavidin molecule (shown as white). Non-specific binding was accounted for by testing arteries not exposed to DS-SILY, with no evident staining.

All delivery methods showed effective delivery to the vessel wall, indicating that collagen was exposed during fistula creation due to endothelial denudation. Blood flow was restored for approximately 2 hours prior to sacrifice in these studies, indicating that the DS-SILY was able to remain bound to the vessel wall during this time.

### Example 3. DS-SILY Prevented Fistula Stenosis

This example performed CT scans on all animals prior to sacrifice at 28 days from example 1. CT is a common method to determine vessel diameter, and has been used in both preclinical and clinical settings. A contrast dye was injected into the animal during the scan so that the vessels could be visualized and the diameter of the vessel could be measured through the images. A radiologist blinded to the treatment type of DS-SILY or saline control measured the diameter of the vein in the fistula from the anastomosis to greater than 3 cm distal to the anastomosis. The smallest diameter measured was denoted as the most stenotic area in the vessel.

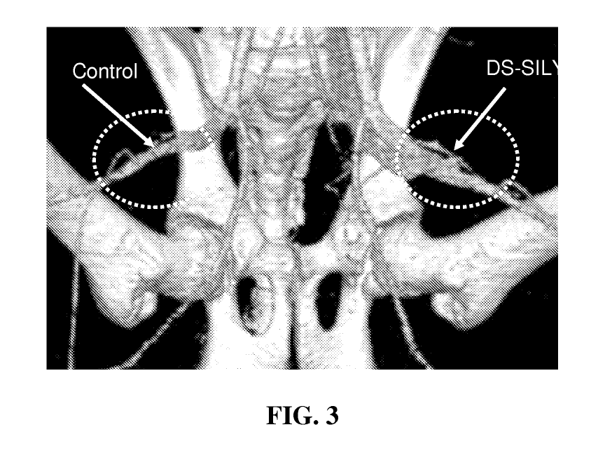

CT images (e.g., FIG. 3) showed that the venous portion of the fistula was completely occluded by 28 days in the control treated fistula, demonstrated by a lack of visible vein due to the inability of contrast dye to flow through the vein. In contrast, the DS-SILY treated fistula remained open throughout the study, and a large vein was evident in the CT scan.

The diameter of the most stenotic region (the smallest portion of the vessel for blood flow) was recorded. In the 4 animals that have been completed to the 28-day timepoint, the average smallest diameter in the control group was 2.1 mm, compared to an average smallest diameter of 6.3 mm for fistulas treated with DS-SILY.

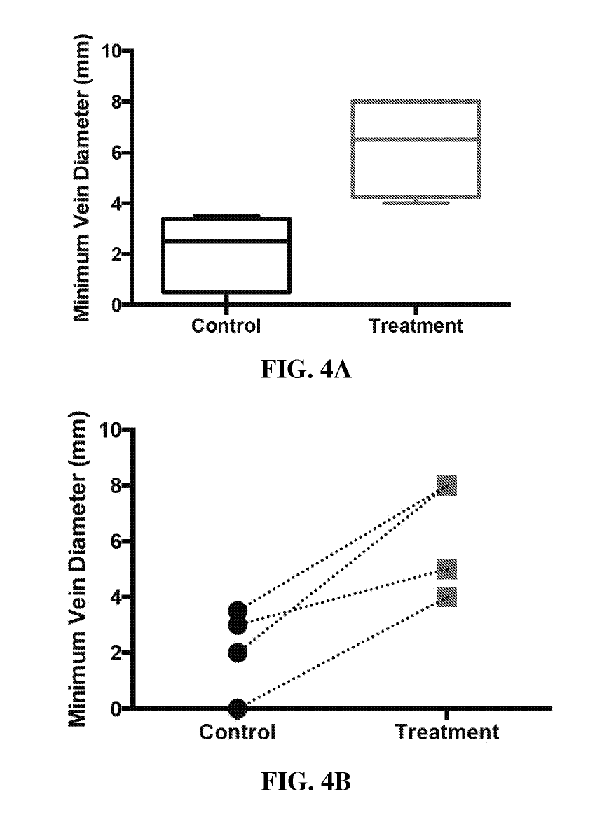

It was surprising that the DS-SILY treated veins had diameters 3 times the size of fistulas treated only with saline. The data is statistically significant with a p-value of 0.019. The data is depicted in FIG. 4A in a box and in FIG. 4B in a whisker plot. In this plot, the top and bottom of the box represent the 1st and 3rd quartile of the data, the line in the middle represents the median point, and the “whiskers” denote the minimum and maximum measurements. The data is also represented in the right most panel showing paired comparisons of the smallest diameter point in the fistula of each animal, either treated with DS-SILY or saline control.

### Example 4. Vein Grafts Treated with DS-SILY

This example will confirm that vein grafts treated with DS-SILY result in reduced vein graft failure when the grafts are used in a bypass surgery. The example will also test for conditions for preparing the vein grafts.

A. Optimize the Concentration of DS-SILY

Arteries from ex vivo studies on excised rabbit blood vessel tissue will be performed to optimize the drug substance concentration and soak duration prior to starting animal model studies. Information from the ex vivo binding studies will be used to define the soak time, drug substance concentration and formulation buffer to generate a vein graft preservation solution.

First, DS-SILY binding to veins will be quantified to examine effect of DS-SILY concentration and soak time. Excised veins from one rabbit will be cut into approximately 1 cm² segments and placed in a 24-well plate. Varying concentrations of DS-SILY in buffered saline and varying times of treatment will be tested. Tissue pieces will be homogenized in extraction medium containing detergents and centrifuged to pellet debris. The supernatant will be assayed for protein by bicinchoninic acid assay (BCA) reagent and for drug substance by ELISA or ECL (electrochemiluminescent technology by MSD, Meso Scale Discovery).

Additionally, to determine how extensive vessel wall damage can enhance the binding, a second set of experiments will be included in which the vessels are scraped gently with a rubber policeman before cutting them into pieces. This procedure will simulate the process in which surgeons remove valves from the vein prior to implantation.

In addition to quantification of DS-SILY as described above, immunohistochemistry (IHC) will be performed to confirm that the drug substance binds to the lumen of the vein. Two conditions (concentration and soak time) will be chosen based on the above experiments for testing. The jugular veins will be excised from a rabbit and flushed and soaked with DS-SILY solution. The veins will then be rinsed in 3 changes of buffered saline. The tissue will be cut into 3 segments. One segment will be fixed in neutral buffered formalin (NBF), a second segment will be cryopreserved in optimal cutting temperature (OCT) and a third will be snap frozen. Tissue sections from cryostat or formalin-fixed, paraffin-embedded (FFPE) specimens will be stained with H&E and immuno stained using antibody specific for drug substance that has already been prepared. While the DS-SILY will bind to any exposed collagen on the vessel, this example expects to see the drug substance coating the inner surface of the blood vessel.

Further, The IHC procedure will be performed using human cadaver vein to ensure translation of procedures to human tissue. Vein tissue will be obtained from cadavers within 24 hours of death.

B. Evaluate the DS-SILY Solution in Vein Bypass Animal Procedure

A vein graft model will be performed in male New Zealand White rabbits. The vein bypass graft will be constructed with an anastomotic cuff technique. The external jugular vein will be harvested and placed in a solution of either heparinized saline or a vein graft preservation solution at the appropriate time and concentration of DS-SILY. Following the storage period, the vein ends will be passed through a polyurethane cuff fashioned from a 4F vascular introducer sheath and then everted over the outside of the cuffs and secured with sutures. The carotid artery lumen will then be exposed with a 1-2 cm arteriotomy. The cuffed and reversed vein ends will be inserted into the carotid arterial lumen and the artery will be secured around the cuff with sutures. Once flow is restored, the interposed segment of the artery will be completely divided to allow full vein graft extension. Graft patency will be confirmed by visualization of pulsatile flow within the graft.

A total of 20 animals will be used. Ten animals will have the vein soaked in heparinized saline prior to grafting into the carotid, and ten animals will have the vein soaked in the vein graft preservation solution. The animals will be survived for 28 days. Heparinized saline is chosen as the control arm because it is commonly used in a clinical setting.

At day 28, anesthesia will be induced and the patency of the vessel will be confirmed. An intravenous heparin bolus will be given, and animals will be sacrificed. Vein grafts will undergo in situ perfusion fixation from the ascending aorta with 10% neutral-buffered formalin (NBF). The vein graft will then be excised and submersion fixed in NBF, prior to paraffin embedding for sectioning and morphometry. At least three different sections of the vein graft will be analyzed along the length of the vein graft, avoiding the tissue immediately adjacent to the foreign body cuffs.

Paraffin embedded 6 μm sections will be stained with Movat's pentachrome stain and imaged with an Aperio microscope. The circumference of the lumen, internal elastic lamina (IEL) and external elastic lamina (EEL) will be outlined and the areas within each perimeter will be calculated. The neointimal area will be calculated as the IEL area—Lumen area.

In each group there are 10 vein grafts, and a minimum of three areas will be examined within each graft. The three measurements within each graft will be averaged to give a mean neointimal area value per graft. The sample size of 10 results in sufficient power (0.8, α=0.05) for detecting a 25% reduction in neointimal hyperplasia with 20% standard deviation in the measurement. The criteria for success in this aim include a 25% reduction in neointimal hyperplasia in vein grafts treated with DS-SILY.

C. Liquid Stability Study.

The design and synthesis of the DS-SILY compound is in the process of being developed with well-established controls. A stability program will be completed to determine the stability of DS-SILY stored as a liquid at room temperature and at 4° C. The DS-SILY solution has previously been stored in a lyophilized form, and reconstituted prior to use. Such a solution was found to be stable for 3 months (time tested to date). Thus, a formal stability protocol for DS-SILY stored as a liquid at 4° C. and at room temperature will be initiated. A liquid formulation would be convenient form of DS-SILY for clinical delivery in this setting.

D. Toxicity Studies in Rats

The dose range finding studies will be performed. First, rats will be given a single IV injection at four dose levels to ascertain acute toxicity after two days. Satellite groups will be similarly dosed and blood samples taken at time intervals to determine pharmacokinetic parameters. Next, a 7-day repeat dose study will be performed in a non-GLP setting in rats with no recovery period. The results from this study will be used to choose a highest tolerable dose level. Finally, a chronic 28-day repeat dose study will be performed with a 28-day recovery period using the highest tolerable dose level.

Endpoints for the studies will include mortality, clinical observations, body weights prior to dosing and at necropsy, food consumption prior to dosing and at necropsy, toxicokinetic observations from blood collection and clinical pathology. Standard histopathology will be performed on standard organs.

It is anticipated that no toxicity will be observed. It is expected that solubility limits will be reached prior to finding any toxic effects.

1. A method for establishing a vascular access in a patient, comprising:
applying a solution to a wall of a blood vessel in a vascular access; and
restoring or initiating blood flow in the vascular access,
wherein the solution comprises an effective amount of a synthetic proteoglycan comprising a glycan having from about 1 to about 80 collagen-binding peptide(s) bonded to the glycan.

2. The method of claim 1, wherein the wall is an internal wall of the blood vessel.

3. The method of claim 1, wherein the vascular access is an arteriovenous fistula (AVF), an arteriovenous graft (AVG), or a durable vascular access used for parenteral nutrition, chemotherapy, or plasmapheresis.

4. The method of claim 1, wherein the solution reduces exposure of the wall to platelets.

5. The method of claim 4, wherein the wall comprises a cell or tissue exposed to blood flow due to injury or a surgical procedure.

6. A method for improving maturation of an arteriovenous fistula (AVF) in a patient in need of hemodialysis, comprising:
applying a solution to the internal wall of a lumen of an AVF; and
restoring or initiating blood flow in the AVF,
wherein the solution comprises an effective amount of a synthetic proteoglycan comprising a glycan having from about 1 to about 80 collagen-binding peptide(s) bonded to the glycan.

7. The method of claim 6, wherein the solution is applied less than about 10 minutes before the blood flow is initiated.

8. The method of claim 7, wherein the solution is flushed through the AVF.

9. The method of claim 8, further comprising closing the AVF after the AVF is flushed with the solution.

10. The method of claim 6, wherein the blood flow is blocked by clamping the proximal vein and artery of an established AVF to form an enclosed lumen, and wherein the solution is injected to the enclosed lumen.

11. The method of claim 6, wherein the solution is applied within about 5 minutes following dilating or rubbing the vein portion of the AVF.

12. The method of claim 6, wherein the total amount of the solution applied is from about 1 ml to about 10 ml.

13. The method of claim 6, wherein the method improves patency, improves survival, improves blood flow, enlarges vascular inner diameter, or reduce stenosis of the AVF.

14. The method of claim 6, wherein the glycan is dextran, chondroitin, chondroitin sulfate, dermatan, dermatan sulfate, heparan sulfate, heparin, keratin, keratan sulfate, or hyaluronic acid.

15. The method of claim 6, wherein the peptide(s) are covalently bonded to the glycan via a linker.

16. The method of claim 15, wherein the linker is N-[β-maleimidopropionic acid]hydrazide (BMPH), 3-(2-pyridyldithio)propionyl hydrazide (PDPH) or the peptide GSG.

17. The method of claim 6, wherein the synthetic proteoglycan comprises from about 5 to about 40 peptides.

18. The method of claim 6, wherein the collagen-binding peptide comprises an amino acid sequence selected from:
i)
(SEQ ID NO: 1)
RRANAALKAGELYKSILY,
(SEQ ID NO: 2)
RLDGNEIKR,
(SEQ ID NO: 3)
AHEEISTTNEGVM,
(SEQ ID NO: 4)
GELYKSILY,
(SEQ ID NO: 5)
NGVFKYRPRYFLYKHAYFYPPLKRFPVQ,
(SEQ ID NO: 6)
CQDSETRTFY,
(SEQ ID NO: 7)
TKKTLRT,
(SEQ ID NO: 8)
GLRSKSKKFRRPDIQYPDATDEDITSHM,
(SEQ ID NO: 9)
SQNPVQP,
(SEQ ID NO: 10)
SYIRIADTNIT,
(SEQ ID NO: 11)
KELNLVYT,
(SEQ ID NO: 12)
GSITTIDVPWNVGC,
(SEQ ID NO: 13)
GSITTIDVPWNV;
(SEQ ID NO: 14)
RRANAALKAGELYKCILY,
(SEQ ID NO: 15)
GELYKCILY,
(SEQ ID NO: 16)
GQLYKSILY,
or
(SEQ ID NO: 17)
RRANAALKAGQLYKSILY
or
ii) a peptide comprising a sequence with at least about 80% sequence identity to the amino acid sequence of i) and capable of binding to collagen.

19. The method of claim 6, wherein the solution is formulated as a film, gel, patch, or liquid solution.

20. The method of claim 19, wherein the solution further comprises a polymer matrix.

21. A method for preparing a vascular graft for a bypass surgery, comprising contacting the internal wall of a section of a blood vessel with a solution comprising an effective amount of a synthetic proteoglycan comprising a glycan having from about 1 to about 80 collagen-binding peptide(s) bonded to the glycan.

22. The method of claim 21, wherein the blood vessel is a vein.

23. The method of claim 21, wherein the contacting is carried out under conditions to allow the synthetic proteoglycan to bind to the internal wall.

24. A vascular graft comprising a section of a blood vessel comprising an internal wall bound to an effective amount of a synthetic proteoglycan comprising a glycan having from about 1 to about 80 collagen-binding peptide(s) bonded to the glycan.

25. A method for preventing or reducing graft failure in a patient undergoing a bypass grafting procedure, comprising implanting a graft into the circulation system of the patient, wherein the graft comprises an internal wall bound to an effective amount of a synthetic proteoglycan comprising a glycan having from about 1 to about 80 collagen-binding peptide(s) bonded to the glycan.

26. A method for preventing or reducing graft failure in a patient undergoing a bypass grafting procedure, comprising implanting a graft into the circulation system of the patient, and injecting into the inside of the graft, before, during or following the implantation, a solution comprising an effective amount of a synthetic proteoglycan comprising a glycan having from about 1 to about 80 collagen-binding peptide(s) bonded to the glycan.

27. The method of claim 26, wherein the collagen-binding peptide(s) is RRANAALKAGELYKSILY (SEQ ID NO: 1).

28. The method of claim 27, wherein the glycan is dermatan sulfate.

29. The method of claim 27, wherein the glycan is heparin.


SEQUENCE LISTING (85 sequences)

SEQ ID NO: 1 - protein, 18 aa, Artificial Sequence
  Description of Artificial Sequence Synthetic peptide
RRANAALKAGELYKSILY

SEQ ID NO: 2 - protein, 9 aa, Artificial Sequence
  Description of Artificial Sequence Synthetic peptide
RLDGNEIKR

SEQ ID NO: 3 - protein, 13 aa, Artificial Sequence
  Description of Artificial Sequence Synthetic peptide
AHEEISTTNEGVM

SEQ ID NO: 4 - protein, 9 aa, Artificial Sequence
  Description of Artificial Sequence Synthetic peptide
GELYKSILY

SEQ ID NO: 5 - protein, 28 aa, Artificial Sequence
  Description of Artificial Sequence Synthetic peptide
NGVFKYRPRYFLYKHAYFYPPLKRFPVQ

SEQ ID NO: 6 - protein, 10 aa, Artificial Sequence
  Description of Artificial Sequence Synthetic peptide
CQDSETRTFY

SEQ ID NO: 7 - protein, 7 aa, Artificial Sequence
  Description of Artificial Sequence Synthetic peptide
TKKTLRT

SEQ ID NO: 8 - protein, 28 aa, Artificial Sequence
  Description of Artificial Sequence Synthetic peptide
GLRSKSKKFRRPDIQYPDATDEDITSHM

SEQ ID NO: 9 - protein, 7 aa, Artificial Sequence
  Description of Artificial Sequence Synthetic peptide
SQNPVQP

SEQ ID NO: 10 - protein, 11 aa, Artificial Sequence
  Description of Artificial Sequence Synthetic peptide
SYIRIADTNIT

SEQ ID NO: 11 - protein, 8 aa, Artificial Sequence
  Description of Artificial Sequence Synthetic peptide
KELNLVYT

SEQ ID NO: 12 - protein, 14 aa, Artificial Sequence
  Description of Artificial Sequence Synthetic peptide
GSITTIDVPWNVGC

SEQ ID NO: 13 - protein, 12 aa, Artificial Sequence
  Description of Artificial Sequence Synthetic peptide
GSITTIDVPWNV

SEQ ID NO: 14 - protein, 18 aa, Artificial Sequence
  Description of Artificial Sequence Synthetic peptide
RRANAALKAGELYKCILY

SEQ ID NO: 15 - protein, 9 aa, Artificial Sequence
  Description of Artificial Sequence Synthetic peptide
GELYKCILY

SEQ ID NO: 16 - protein, 9 aa, Artificial Sequence
  Description of Artificial Sequence Synthetic peptide
GQLYKSILY

SEQ ID NO: 17 - protein, 18 aa, Artificial Sequence
  Description of Artificial Sequence Synthetic peptide
RRANAALKAGQLYKSILY

SEQ ID NO: 18 - protein, 10 aa, Artificial Sequence
  Description of Artificial Sequence Synthetic peptide
WREPSFCALS

SEQ ID NO: 19 - protein, 16 aa, Artificial Sequence
  Description of Artificial Sequence Synthetic peptide
  MOD_RES (1)..(1): Beta-alanine
  MOD_RES (16)..(16): Biphenylalanine
AWHCTTKFPHHYCLYF

SEQ ID NO: 20 - protein, 17 aa, Artificial Sequence
  Description of Artificial Sequence Synthetic peptide
  MOD_RES (1)..(1): Beta-alanine
  MOD_RES (17)..(17): Biphenylalanine
AHKCPWHLYTTHYCFTF

SEQ ID NO: 21 - protein, 15 aa, Artificial Sequence
  Description of Artificial Sequence Synthetic peptide
  MOD_RES (1)..(1): Beta-alanine
AHKCPWHLYTHYCFT

SEQ ID NO: 22 - protein, 6 aa, Artificial Sequence
  Description of Artificial Sequence Synthetic peptide
  MOD_RES (3)..(3): 4-hydroxyproline
GRPGER

SEQ ID NO: 23 - protein, 6 aa, Artificial Sequence
  Description of Artificial Sequence Synthetic peptide
  MOD_RES (3)..(3): 4-hydroxyproline
GMPGER

SEQ ID NO: 24 - protein, 6 aa, Artificial Sequence
  Description of Artificial Sequence Synthetic peptide
  MOD_RES (3)..(3): 4-hydroxyproline
GLPGEN

SEQ ID NO: 25 - protein, 6 aa, Artificial Sequence
  Description of Artificial Sequence Synthetic peptide
  MOD_RES (3)..(3): 4-hydroxyproline
GLPGER

SEQ ID NO: 26 - protein, 6 aa, Artificial Sequence
  Description of Artificial Sequence Synthetic peptide
GLKGEN

SEQ ID NO: 27 - protein, 15 aa, Artificial Sequence
  Description of Artificial Sequence Synthetic peptide
  MOD_RES (3)..(3): 4-hydroxyproline
  MOD_RES (12)..(12): 4-hydroxyproline
GFPGERGVEGPPGPA

SEQ ID NO: 28 - protein, 11 aa, Artificial Sequence
  Description of Artificial Sequence Synthetic peptide
HVWMQAPGGGK

SEQ ID NO: 29 - protein, 6 aa, Artificial Sequence
  Description of Artificial Sequence Synthetic peptide
WYRGRL

SEQ ID NO: 30 - protein, 12 aa, Artificial Sequence
  Description of Artificial Sequence Synthetic peptide
WTCSGDEYTWHC

SEQ ID NO: 31 - protein, 12 aa, Artificial Sequence
  Description of Artificial Sequence Synthetic peptide
WTCVGDHKTWKC

SEQ ID NO: 32 - protein, 16 aa, Artificial Sequence
  Description of Artificial Sequence Synthetic peptide
QWHCTTRFPHHYCLYG

SEQ ID NO: 33 - protein, 17 aa, Artificial Sequence
  Description of Artificial Sequence Synthetic peptide
STWTWNGSAWTWNEGGK

SEQ ID NO: 34 - protein, 17 aa, Artificial Sequence
  Description of Artificial Sequence Synthetic peptide
STWTWNGTNWTRNDGGK

SEQ ID NO: 35 - protein, 8 aa, Artificial Sequence
  Description of Artificial Sequence Synthetic peptide
CVWLWEQC

SEQ ID NO: 36 - protein, 8 aa, Artificial Sequence
  Description of Artificial Sequence Synthetic peptide
CMTSPWRC

SEQ ID NO: 37 - protein, 18 aa, Artificial Sequence
  Description of Artificial Sequence Synthetic peptide
CPGRVMHGLHLGDDEGPC

SEQ ID NO: 38 - protein, 7 aa, Artificial Sequence
  Description of Artificial Sequence Synthetic peptide
KLWLLPK

SEQ ID NO: 39 - protein, 9 aa, Artificial Sequence
  Description of Artificial Sequence Synthetic peptide
LSELRLHEN

SEQ ID NO: 40 - protein, 9 aa, Artificial Sequence
  Description of Artificial Sequence Synthetic peptide
LTELHLDNN

SEQ ID NO: 41 - protein, 9 aa, Artificial Sequence
  Description of Artificial Sequence Synthetic peptide
LSELRLHNN

SEQ ID NO: 42 - protein, 9 aa, Artificial Sequence
  Description of Artificial Sequence Synthetic peptide
LSELRLHAN

SEQ ID NO: 43 - protein, 9 aa, Artificial Sequence
  Description of Artificial Sequence Synthetic peptide
LRELHLNNN

SEQ ID NO: 44 - protein, 12 aa, Artificial Sequence
  Description of Artificial Sequence Synthetic peptide
RVMHGLHLGDDE

SEQ ID NO: 45 - protein, 12 aa, Artificial Sequence
  Description of Artificial Sequence Synthetic peptide
  MOD_RES (1)..(12): D-amino acids
EDDGLHLGHMVR

SEQ ID NO: 46 - protein, 12 aa, Artificial Sequence
  Description of Artificial Sequence Synthetic peptide
RVMHGLHLGNNQ

SEQ ID NO: 47 - protein, 12 aa, Artificial Sequence
  Description of Artificial Sequence Synthetic peptide
  MOD_RES (1)..(12): D-amino acids
QNNGLHLGHMVR

SEQ ID NO: 48 - protein, 12 aa, Artificial Sequence
  Description of Artificial Sequence Synthetic peptide
RVMHGLHLGNNQ

SEQ ID NO: 49 - protein, 12 aa, Artificial Sequence
  Description of Artificial Sequence Synthetic peptide
GQLYKSILYGSG

SEQ ID NO: 50 - protein, 11 aa, Artificial Sequence
  Description of Artificial Sequence Synthetic peptide
GSGQLYKSILY

SEQ ID NO: 51 - protein, 12 aa, Artificial Sequence
  Description of Artificial Sequence Synthetic peptide
GSGGQLYKSILY

SEQ ID NO: 52 - protein, 8 aa, Artificial Sequence
  Description of Artificial Sequence Synthetic peptide
KQLNLVYT

SEQ ID NO: 53 - protein, 8 aa, Artificial Sequence
  Description of Artificial Sequence Synthetic peptide
CVWLWQQC

SEQ ID NO: 54 - protein, 10 aa, Artificial Sequence
  Description of Artificial Sequence Synthetic peptide
WREPSFSALS

SEQ ID NO: 55 - protein, 28 aa, Artificial Sequence
  Description of Artificial Sequence Synthetic peptide
GHRPLDKKREEAPSLRPAPPPISGGGYR

SEQ ID NO: 56 - protein, 28 aa, Artificial Sequence
  Description of Artificial Sequence Synthetic peptide
GHRPLNKKRQQAPSLRPAPPPISGGGYR

SEQ ID NO: 57 - protein, 28 aa, Artificial Sequence
  Description of Artificial Sequence Synthetic peptide
GELYKSILYGSGGELYKSILYGSGKGSG

SEQ ID NO: 58 - protein, 41 aa, Artificial Sequence
  Description of Artificial Sequence Synthetic polypeptide
GELYKSILYGSGGELYKSILYGSGGELYKSILYGSGKKGSG

SEQ ID NO: 59 - protein, 54 aa, Artificial Sequence
  Description of Artificial Sequence Synthetic polypeptide
GELYKSILYGSGGELYKSILYGSGGELYKSILYGSGGELYKSILYGSGKKKGSG

SEQ ID NO: 60 - protein, 28 aa, Artificial Sequence
  Description of Artificial Sequence Synthetic peptide
GQLYKSILYGSGGQLYKSILYGSGKGSG

SEQ ID NO: 61 - protein, 41 aa, Artificial Sequence
  Description of Artificial Sequence Synthetic polypeptide
GQLYKSILYGSGGQLYKSILYGSGGQLYKSILYGSGKKGSG

SEQ ID NO: 62 - protein, 54 aa, Artificial Sequence
  Description of Artificial Sequence Synthetic polypeptide
GQLYKSILYGSGGQLYKSILYGSGGQLYKSILYGSGGQLYKSILYGSGKKKGSG

SEQ ID NO: 63 - protein, 13 aa, Artificial Sequence
  Description of Artificial Sequence Synthetic peptide
RYPISRPRKRGSG

SEQ ID NO: 64 - protein, 20 aa, Artificial Sequence
  Description of Artificial Sequence Synthetic peptide
RRANAALKAGELYKSILYGC

SEQ ID NO: 65 - protein, 11 aa, Artificial Sequence
  Description of Artificial Sequence Synthetic peptide
GELYKSILYGC

SEQ ID NO: 66 - protein, 11 aa, Artificial Sequence
  Description of Artificial Sequence Synthetic peptide
RLDGNEIKRGC

SEQ ID NO: 67 - protein, 15 aa, Artificial Sequence
  Description of Artificial Sequence Synthetic peptide
AHEEISTTNEGVMGC

SEQ ID NO: 68 - protein, 12 aa, Artificial Sequence
  Description of Artificial Sequence Synthetic peptide
GCGGELYKSILY

SEQ ID NO: 69 - protein, 30 aa, Artificial Sequence
  Description of Artificial Sequence Synthetic polypeptide
NGVFKYRPRYFLYKHAYFYPPLKRFPVQGC

SEQ ID NO: 70 - protein, 12 aa, Artificial Sequence
  Description of Artificial Sequence Synthetic peptide
CQDSETRTFYGC

SEQ ID NO: 71 - protein, 9 aa, Artificial Sequence
  Description of Artificial Sequence Synthetic peptide
TKKTLRTGC

SEQ ID NO: 72 - protein, 30 aa, Artificial Sequence
  Description of Artificial Sequence Synthetic polypeptide
GLRSKSKKFRRPDIQYPDATDEDITSHMGC

SEQ ID NO: 73 - protein, 9 aa, Artificial Sequence
  Description of Artificial Sequence Synthetic peptide
SQNPVQPGC

SEQ ID NO: 74 - protein, 13 aa, Artificial Sequence
  Description of Artificial Sequence Synthetic peptide
SYIRIADTNITGC

SEQ ID NO: 75 - protein, 10 aa, Artificial Sequence
  Description of Artificial Sequence Synthetic peptide
KELNLVYTGC

SEQ ID NO: 76 - protein, 14 aa, Artificial Sequence
  Description of Artificial Sequence Synthetic peptide
GCGGELYKSILYGC

SEQ ID NO: 77 - protein, 21 aa, Artificial Sequence
  Description of Artificial Sequence Synthetic peptide
  C-term NHNH2
RRANAALKAGELYKSILYGSG

SEQ ID NO: 78 - protein, 4 aa, Artificial Sequence
  Description of Artificial Sequence Synthetic peptide
GGGG

SEQ ID NO: 79 - protein, 5 aa, Artificial Sequence
  Description of Artificial Sequence Synthetic peptide
GGGGG

SEQ ID NO: 80 - protein, 4 aa, Artificial Sequence
  Description of Artificial Sequence Synthetic peptide
GSGC

SEQ ID NO: 81 - protein, 5 aa, Artificial Sequence
  Description of Artificial Sequence Synthetic peptide
GSGSG

SEQ ID NO: 82 - protein, 4 aa, Artificial Sequence
  Description of Artificial Sequence Synthetic peptide
KGSG

SEQ ID NO: 83 - protein, 5 aa, Artificial Sequence
  Description of Artificial Sequence Synthetic peptide
KKGSG

SEQ ID NO: 84 - protein, 6 aa, Artificial Sequence
  Description of Artificial Sequence Synthetic peptide
KKKGSG

SEQ ID NO: 85 - protein, 4 aa, Artificial Sequence
  Description of Artificial Sequence Synthetic peptide
GGGC